(19) United States
(12) Patent Application Publication
(10) Pub. No.: US 2023/0078647 A1
(43) Pub. Date: Mar. 16, 2023

(54) SYSTEMS, APPARATUS, AND METHODS FOR TREATMENT OF VARICOCELE AND ASSOCIATED CONDITIONS

(71) Applicant: Vivifi Medical LLC, Rosharon, TX (US)

(72) Inventors: Tushar SHARMA, Rosharon, TX (US); Nicolo GARBIN, Houston, TX (US)

(73) Assignee: Vivifi Medical LLC, Rosharon, TX (US)

(21) Appl. No.: 17/824,511

(22) Filed: May 25, 2022

Related U.S. Application Data
(63) Continuation of application No. PCT/US2020/062287, filed on Nov. 25, 2020.
(60) Provisional application No. 62/940,595, filed on Nov. 26, 2019.

(51) Int. Cl. A61B 17/12

(52) U.S. Cl. CPC A61B 17/12109; A61B 17/12022; A61B 17/12136

(57) ABSTRACT

Systems, apparatus, and methods are described for treatment of varicocele and associated conditions. In some embodiments, systems, apparatus, and methods described herein can include forming one or more fluid connections or fistulas between blood vessels such as a gonadal vein (e.g., spermatic vein, ovarian vein) and surrounding veins. In some embodiments, systems, apparatus, and methods described herein can include occluding one or more blood vessels such as a gonadal vein (e.g., spermatic vein, ovarian vein). In some embodiments, systems, apparatus and methods described herein relate to flow diverters, replacement valves, etc., e.g., for treatment of varicocele and associated conditions.

### CROSS-REFERENCE TO RELATED APPLICATIONS

This application is a continuation of International Patent Application No. PCT/US2020/062287, entitled “Systems, Apparatus, and Methods for Treatment of Varicocele and Associated Conditions,” filed Nov. 25, 2020 which claims priority to U.S. Provisional Application No. 62/940,595, entitled “Treatment of Blood Vessel Related Conditions,” filed Nov. 26, 2019, the disclosure of each of which is incorporated herein by reference.

### TECHNICAL FIELD

The present disclosure relates generally to systems, apparatus, and methods for treatment of varicocele, erectile dysfunction, infertility, nutcracker syndrome, benign prostate hyperplasia (BPH), bladder cancer, prostate cancer, pelvic congestion, ovarian cancer, polycystic ovarian syndrome, uterine fibroids, endometriosis and/or hormonal disorders in relation to testosterone.

### BACKGROUND

As depicted in FIG. 1, the left internal spermatic vein (ISV) merges with the left renal vein at a right angle near the superior mesenteric artery, while the right internal spermatic vein tends to merge with the inferior vena cava at more of an acute angle. One-way valves within the ISVs prevent back-flow of venous blood draining from the testicles. Venous blood from the testicles must flow upwards against gravitational forces that are the highest while standing. The repetitive loading of the one-way valves in the ISVs (a consequence of the bipedal, or upright, posture in humans) frequently leads to gradual deterioration and eventual failure of the valves. Faulty, non-functional one-way valves lead to impaired blood flow, or even a reflux, of venous blood from the testes. Without competent one-way valves, the ISVs cease to properly function as drainage systems and instead act as hydrostatic columns of blood that, particularly when standing, exert a back-pressure on the testicular venous drainage system. The hydrostatic pressure exerted by the ISVs often exceeds that of testicular venous blood vessels located inferiorly to the ISVs, preventing physiological fluid flow through the ISVs. Chronic reduced blood flow from the testes can result in the development of a pathophysiological condition called varicocele.

Reduced testicular blood flow causes persistent hypoxia in the testicular microcirculation, leading to deterioration in spermatogenesis and insufficient testosterone production. As a result of anatomical differences in vessel length (amongst other geometric differences), varicocele is more common in the left ISV than the right ISV. Consequently, varicocele of the left ISV is more readily diagnosed and has been widely linked to male infertility in the medical literature. Additionally, the ISVs comprise a network of small bypasses and retroperitoneal collaterals. Each of these, when oriented vertically, similarly create pathophysiological hydrostatic pressure in the pampiniform plexus (PP).

The development of varicocele can also be caused by the compression of the renal vein by the superior mesenteric artery and the aorta, also called the nutcracker phenomenon (NCP) or left renal vein entrapment. FIG. 2 depicts a normal renal vein (left) alongside a compressed renal vein (right). Varicocele is associated with the left ISV in 95% of the cases, and the prevalence of varicocele has been shown to increase with age, reaching over 75% at the age of 70. Several studies have demonstrated a strong correlation between varicocele and left renal vein compression. Several studies have also demonstrated that the destruction of one-way valves can lead to bilateral vascular disease.

FIG. 3 depicts a synopsis of the progression of varicocele and its associated complications (e.g., diseases and medical conditions). Recent studies have suggested a linkage between varicocele and low serum testosterone levels. Although varicocele has been theorized to be connected with testosterone levels, and testosterone has been shown to play a role in prostate cancer, there has been no established causal correlation between varicocele and prostate disease, and, paradoxically, relatively low levels of serum testosterone were found in patients with prostate cancer and benign prostatic hyperplasia.

Benign Prostatic Hyperplasia (BPH) is one of the most common medical conditions that affects men, especially in elderly men. It has been reported that more than half of all men have histopathologic evidence of BPH by age 60 and, by age 85, approximately 9 out of 10 men suffer from the condition. Moreover, the incidence and prevalence of BPH are expected to increase as the average age of the global population increases. Although BPH is rarely life threatening, it can lead to numerous clinical conditions including urinary retention, renal insufficiency, recurrent urinary tract infection, incontinence, hematuria, and bladder stones. It has been estimated that by the age of 80 years, approximately 25% of the male population of the United States will have undergone some form of treatment for the complications associated with BPH. The currently available treatment options for BPH include watchful waiting, medications (phytotherapy and prescription medications), surgery, and minimally invasive procedures.

Recent studies have shown a link between BPH, low systemic testosterone levels, and varicocele. Hypertension has also been linked to the development of BPH, reinforcing the theory that renal vein compression can lead to the development of varicocele(s) and eventually BPH.

Chronic inflammation has been shown to be an important factor in the development and progression of BPH. Chronic inflammation of the prostate can lead to over-exposure to high levels of free testosterone. As a result of chronic insufficient testicular circulation from varicocele(s), the prostate is exposed to concentrations of bioactive testosterone exceeding more than 100 times normal serum levels, resulting in accelerated cell proliferation. The abnormally accelerated rate of cell proliferation in the prostate leads to errors in DNA replication that can eventually lead to the development of prostate cancer. In addition to being linked to BPH and prostate cancer, varicocele has been shown to cause infertility, and studies have also shown a link between infertility and an increased risk of several types of cancers.

Although varicocele is a disease commonly associated with men, varicocele of the ovarian vein(s) in women have been linked to ovarian vein syndrome, pelvic congestion syndrome chronic pelvic pain, pelvic varicosities, vulvar varicosities and varicosities of the lower limbs. Similarly to the progression of varicocele(s) in males, deterioration of the one-way valves in the ovarian veins in females, clinically referred to as pelvic congestion (PCS), may lead to impaired ovarian blood flow, or even reflux of venous blood into the ovaries and uterine venous plexus. Several studies have also shown an association between pelvic varices and polycystic ovarian syndrome, suggesting excessive oestrogen secretion may be a potential cause of the development of these diseases. Coil embolization of the ovarian veins in patients with pelvic congestion syndrome or varicocele and demonstrable pelvic varicoceles has resulted in a reduction in symptom severity in 56% to 98% of patients.

Analysis of the available literature shows a need for a novel, more effective treatment to reduce, eliminate, and/or prevent pathophysiological hydrostatic pressure in the testicular venous drainage system. Existing treatments are ineffective and can be associated with various drawbacks.

### SUMMARY

Systems, apparatus, and methods are described for treatment of varicocele, erectile dysfunction, infertility, nutcracker syndrome, BPH, bladder cancer, prostate cancer, pelvic congestion, hormonal disorders, or other medical diseases or conditions associated with high blood pressure in the spermatic or ovarian vein(s).

A commonality of some embodiments described herein relates to the linkage between high blood pressure in the spermatic or ovarian vein(s) and the development of several prostate, pelvic, and hormonal disorders, which may include varicocele, erectile dysfunction, infertility, nutcracker syndrome, BPH, prostate cancer, pelvic congestion, ovarian cancer, polycystic ovarian syndrome, hypogonadism, and other disorders. By reducing or eliminating the elevated blood pressure in one or more of the spermatic or ovarian veins, the resulting effects and disorders as listed above can be entirely, or part thereof, mitigated.

In some embodiments, a method for the treatment of, at least partially, varicocele, erectile dysfunction, infertility, nutcracker syndrome, benign prostate hyperplasia (BPH), bladder cancer, prostate cancer, pelvic congestion, and/or hormonal disorders includes deployment of a device, or set of devices, that redirect venous blood flow from the venous blood drainage system of the testes.

In some embodiments, a method for the treatment of, at least partially, varicocele, infertility, nutcracker syndrome, benign hyperplasia, bladder cancer, pelvic congestion, ovarian cancer, polycystic ovarian syndrome, uterine fibroids, endometriosis, and/or hormonal disorders includes deployment of a device, or set of devices, that redirect venous blood flow from the venous blood drainage system of the ovaries.

In some embodiments, a method for the treatment of, at least partially, varicocele, erectile dysfunction, infertility, nutcracker syndrome, benign prostate hyperplasia (BPH), bladder cancer, prostate cancer, and/or hormonal disorders includes creation of a fistula between the spermatic vein(s) and another blood vessel.

In some embodiments, a method for the treatment of, at least partially, varicocele, infertility, nutcracker syndrome, bladder cancer, pelvic congestion, ovarian cancer, polycystic ovarian syndrome, uterine fibroids, endometriosis, and/or hormonal disorders includes creation of a fistula between the ovarian vein(s) and another blood vessel.

In some embodiments, a method for the treatment of, at least partially, varicocele, erectile dysfunction, infertility, nutcracker syndrome, benign prostate hyperplasia (BPH), bladder cancer, prostate cancer, and/or hormonal disorders includes ligation of the spermatic vein(s).

In some embodiments, a method for the treatment of, at least partially, varicocele, infertility, bladder cancer, pelvic congestion, ovarian cancer, polycystic ovarian syndrome, uterine fibroids, endometriosis and/or hormonal disorders includes ligation of the ovarian vein(s).

In some embodiments, a method for the treatment of, at least partially, varicocele, erectile dysfunction, infertility, nutcracker syndrome, benign prostate hyperplasia (BPH), bladder cancer, prostate cancer, pelvic congestion, and/or hormonal disorders includes occlusion of the deferential vein(s).

In some embodiments, a method for the treatment of, at least partially, varicocele, erectile dysfunction, infertility, nutcracker syndrome, benign prostate hyperplasia (BPH), bladder cancer, prostate cancer, and/or hormonal disorders includes occlusion of the testicular artery(s).

In some embodiments, a method for the treatment of, at least partially, varicocele, infertility, bladder cancer, pelvic congestion, ovarian cancer, polycystic ovarian syndrome, uterine fibroids, endometriosis and/or hormonal disorders includes occlusion of the ovarian artery(s).

In some embodiments, a system for forming a fluid connection between first and second vessels to treat varicocele and associated conditions, the system includes: a first catheter defining a first channel that terminates in a first aperture, the first catheter including a first alignment element, the first catheter configured to be disposed in the first vessel; a second catheter defining a second channel that terminates in a second aperture, the second catheter including a second alignment element, the second catheter configured to be disposed in the second vessel, the first and second alignment elements configured to align the first and second apertures of the first and second catheters when the first and second catheters are disposed in first and second vessels; at least one piercing element configured to pierce through tissue adjacent to the first and second apertures in a space between the first and second catheters; and a bridging device advanceable through the first channel of the first catheter, through the first and second apertures and the adjacent tissue, and into the second channel of the second catheter such that a portion of the bridging device extends through the space between the first and second catheters, the bridging device configured to allow an implant to be deployed in the space between the first and second catheters to form the fluid connection between the first and second vessels.

In some embodiments, a method for treating varicocele and associated conditions, the method includes: advancing a first catheter into a first vein; advancing a second catheter into a second vein; aligning a first aperture of the first catheter with a second aperture of the second catheter; forming an opening in a wall of the first vein adjacent to the first aperture and an opening a wall of the second vein adjacent to the second aperture; advancing a bridging device from the first catheter, through the openings, and into the second catheter such that a portion of the bridging device extends between the first and second veins; and deploying an implant around the portion of the bridging device extending between the first and second veins.

In some embodiments, a system for forming a fluid connection between first and second veins to treat varicocele and associated conditions, the system includes: a bridging element including a piercing end configured to percutaneously pierce through tissue, the bridging element configured to be advanced through the first vein and into the second vein such that the piercing end forms first and second openings in the first vein and a third opening in the second vein; and a deployment element positionable over the bridging element, the deployment element configured to: deploy an implant that extends between one of the first and second openings and the third opening and forms the fluid connection; and deploy a plug in the other of the first and second openings to prevent hemorrhage of the first vein.

In some embodiments, a method for treating varicocele and associated conditions, the method includes: identifying a target pathway through patient anatomy that intersects first and second veins; advancing a bridging element including a piercing end along the pathway such that the bridging element extends through the first vein and into the second vein; positioning a deployment element over the bridging element such that a portion of the deployment element is positioned between the first and second veins; deploying, via the deployment element, an implant between the first and second veins; and deploying, via the deployment element, a plug into a separate opening in at least one of the first and second veins.

In some embodiments, a system for forming a fistula between first and second veins to treat varicocele and associated conditions, the system includes: a first catheter defining a channel that terminates in an aperture, the first catheter including a first alignment element, the first catheter configured to be disposed in the first vein; a second catheter including a second alignment element, the second catheter configured to be disposed in the second vein, the first and second alignment elements configured to align the first and second apertures of the first and second catheters when the first and second catheters are disposed in first and second veins; and an energy delivery element supported by the first catheter, the energy delivery element configured to deliver energy to ablate tissue adjacent to the aperture between the first and second catheters.

In some embodiments, a method for treating varicocele and associated conditions, the method includes: advancing a first catheter into a first vein; advancing a second catheter into a second vein, the second vein neighboring the first vein; aligning a first alignment element of the first catheter with a second alignment element of the second catheter; and applying energy to ablate tissue between the first and second catheters using an energy delivery element supported by the first catheter.

In some embodiments, a method for treating varicocele and associated conditions, the method includes: advancing a catheter within or near to a vein having a damaged valve and incapable of draining blood from patient anatomy; deploying a vessel closure member within or around the vein; and activating an energy delivery element to ablate a portion of a wall of the vein to cause closure of the vein.

In some embodiments, a method for treating varicocele and associated conditions, the method includes: advancing a catheter into a vein having a damaged valve and incapable of draining blood from patient anatomy; maintaining a heat-set spring within the catheter at a first temperature below a predefined threshold temperature, the heat-set spring at the first temperature being in a first configuration; and deploying the heat-set spring around the vein such that the heat-set spring is exposed to a second temperature above the predefined threshold temperature, the heat-set spring deforming into a second configuration to close the vein in response to being at the second temperature.

Other systems, processes, and features will become apparent to those skilled in the art upon examination of the following drawings and detailed description. It is intended that all such additional systems, processes, and features be included within this description, be within the scope of the present invention, and be protected by the accompanying claims.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 illustrates an anatomy of a male showing a spermatic vein and nearby structures.

FIG. 2 illustrates a normal renal vein alongside a compressed renal vein.

FIG. 3 illustrates a pathophysiology of varicocele-related diseases and medical conditions.

FIG. 4 is a schematic diagram of an example device for creating fluid connections between two or more blood vessels and deploying an implant, according to embodiments described herein.

FIG. 5 is a schematic diagram of catheters used with an example device for creating fluid connections between two or more blood vessels, according to embodiments described herein.

FIG. 6 is a flow chart of a method for creating a fluid connection between two or more vessels using an implant, according to embodiments described herein.

FIGS. 7A, 7C, and 7E depict different examples of implants for creating fluid connections implemented as fluid shunts, according to embodiments described herein. FIGS. 7B, 7D, and 7F depict the example implants depicted in FIGS. 7A, 7C, and 7E disposed between two vessels, respectively, according to embodiments described herein.

FIG. 8 depicts a balloon catheter for introducing over a guidewire and deploying a fluid shunt, according to embodiments described herein.

FIG. 9 depicts a bridging device implemented as a bridging guidewire over which a fluid shunt can be positioned, according to embodiments described herein.

FIGS. 10A, 10B, and 10C depict catheters for guiding a bridging device to a deployment site for a fluid shunt, according to embodiments described herein.

FIGS. 10D and 10E depict cross-sectional views of the catheters depicted in FIG. 10C.

FIG. 11 depicts two vessels located within a human body across which a fluid connection can be established, according to embodiments described herein.

FIGS. 12A and 12B depict enlarged view of a portion of the two vessels depicted in FIG. 11.

FIG. 13 depicts a percutaneous bridging device positioned within the two vessels depicted in FIG. 11 to form a fluid connection between the two vessels, according to embodiments described herein.

FIGS. 14A-14C depict a percutaneous approach for creating a fluid connection between two vessels, according to embodiments described herein.

FIGS. 15A and 15B depict different type of implants that can be deployed within or between two vessels to establish a fluid connection between the vessels, according to embodiments described herein.

FIG. 16 depicts an example of an implant implemented as a fluid shunt, according to embodiments described herein.

FIG. 17 depicts a delivery system for deploying a fluid shunt, including visualization elements, according to embodiments described herein.

FIGS. 18A-18D depict a process for deploying a fluid shunt using a delivery system, according to embodiments described herein.

FIG. 19 depicts a laparoscopic and endovascular approach to deploying a fluid shunt between two vessels, according to embodiments described herein.

FIG. 20 depicts a laparoscopic view of the vessels in the scrotum, including the spermatic vein.

FIGS. 21-22 depicts catheters for creating a fluid connection between two or more blood vessels, according to embodiments described herein.

FIGS. 23A-23C depict a process of creating a fluid connection between two or more vessels using a bridging device and a snare element, according to embodiments described herein.

FIG. 24 is a schematic diagram of an example device for creating fluid connections between two or more blood vessels, according to embodiments described herein.

FIG. 25 is a flow chart of a method for creating a fluid connection between two or more vessels, according to embodiments described herein.

FIG. 26 depicts an example device for creating a fluid connection between two or more vessels, according to embodiments described herein.

FIGS. 27A and 27B depict a process of creating a fluid connection between two vessels, according to embodiments described herein.

FIGS. 28A and 28B depict a process of creating a fluid connection between two vessels, according to embodiments described herein.

FIGS. 29A and 29B depict a process of creating a fluid connection between two vessels, according to embodiments described herein.

FIGS. 30A and 30B provide a detailed view of a distal end of an example device for creating a fluid connection between two or more vessels, according to embodiments described herein.

FIG. 31 depicts a guidewire that can be used with devices for creating fluid connections between two or more vessels, according to embodiments described herein.

FIG. 32 depicts a catheter for delivering the guidewire depicted in FIG. 31, according to embodiments described herein.

FIG. 33 depicts a catheter for receiving the guidewire depicted in FIG. 31, according to embodiments described herein.

FIGS. 34A and 34B depict the catheters depicted in FIGS. 32 and 33 being used together to form a fluid connection between two vessels, according to embodiments described herein.

FIG. 35 depicts an example device for creating a fluid connection between two or more vessels, according to embodiments described herein.

FIG. 36 depicts an example of a percutaneous device for creating a fluid connection between two or more vessels, according to embodiments described herein.

FIGS. 37 and 38 depict a process of creating a fluid connection between two vessels using a percutaneous device, according to embodiments described herein.

FIGS. 39A and 39B depict two different configurations of an example device for creating a fluid connection between two or more vessels, according to embodiments described herein.

FIGS. 40A-40C depict a process of creating a fluid connection between two vessels using a percutaneous device, according to embodiments described herein.

FIGS. 41 and 42 depict a device for adjusting a flow rate of blood in the venous system using an endovascular stent, according to embodiments described herein.

FIG. 43 depicts a device for adjusting a flow rate of blood in the venous system using a flow diverter, according to embodiments described herein.

FIG. 44 is a schematic diagram of an example device for occluding or ligating a vessel, according to embodiments described herein.

FIG. 45 is a flow chart of a method for occluding or ligating a vessel, according to embodiments described herein.

FIG. 46 is depicts an example device for inducing embolization of a vessel, according to embodiments described herein.

FIG. 47 depicts a device for inducing embolization of a vessel disposed within a vessel, according to embodiments described herein.

FIGS. 48A and 48B depict a vessel after being treated with a device for inducing embolization, according to embodiments described herein.

FIGS. 49A-49D depict a process of deploying a heat-activated spring for vessel closure, according to embodiments described herein.

FIGS. 50A-50C depict a process of using a intraluminal vessel ligation catheter to close a vessel, according to embodiments described herein.

FIGS. 51A-51C depict a process of using a percutaneous vessel sealing device to close a vessel, according to embodiments described herein.

FIGS. 52A-52C depict a venous one-way valve that can be deployed in a vessel to improve venous blood flow, according to embodiments described herein.

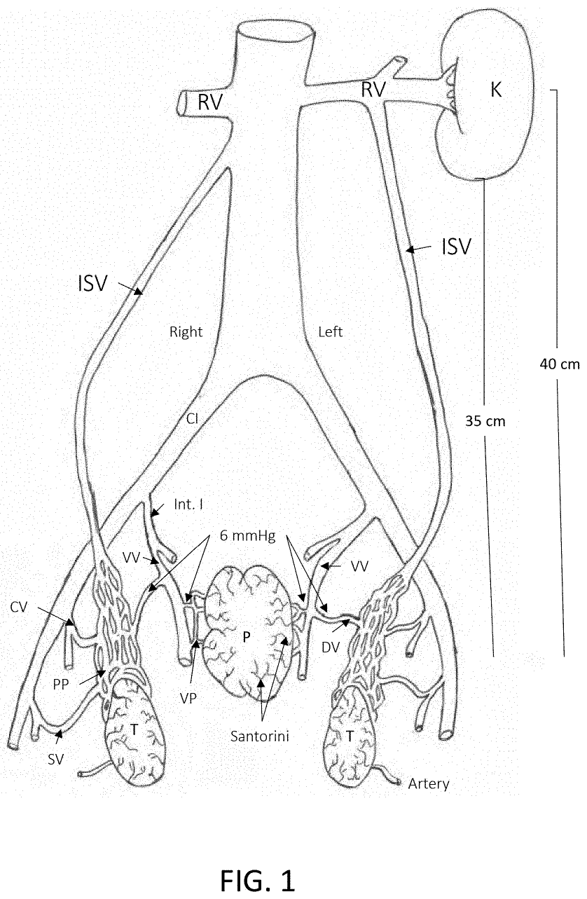

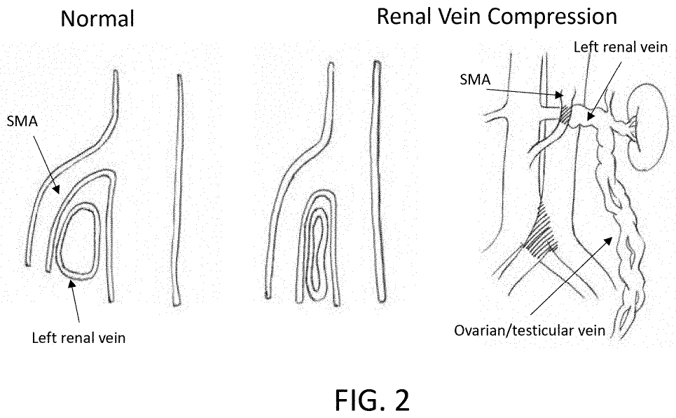

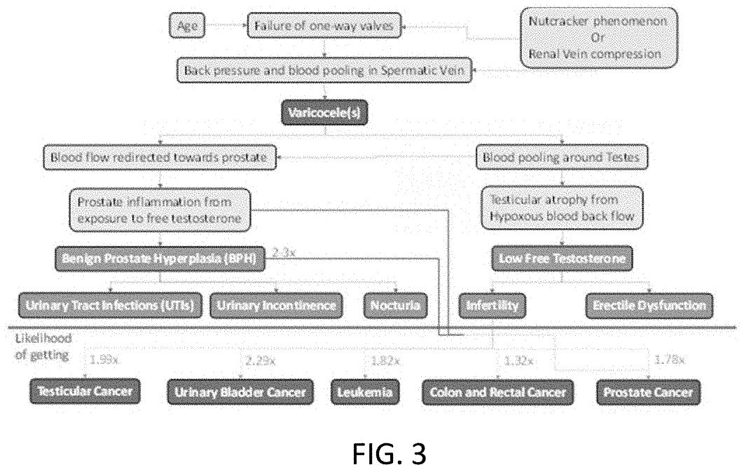

### DETAILED DESCRIPTION

Systems, apparatus, and methods are described herein for treatment of varicocele, erectile dysfunction, infertility, nutcracker syndrome, benign prostate hyperplasia (BPH), bladder cancer, prostate cancer, pelvic congestion, hormonal disorders, or other medical diseases or conditions associated with high blood pressure in the spermatic or ovarian vein(s).

Options for treating varicocele and its associated complications include microsurgery, laparoscopic ligation, and super-selective sclerotherapy of malfunctioning ISVs. These treatments reduce and can eliminate the pressure gradient between the testicular drainage systems, preventing back-flow of blood from the testes to the prostate. The normal physiological functioning of the prostate is restored, with normal venous pressures, a normal arterial blood flow through the prostate, and exposure of the prostate to normal levels of free testosterone.

Microsurgical procedures involve making an incision to access the spermatic cord. The spermatic cord is opened and the veins in the pampiniform plexus are carefully separated from the surrounding arteries, lymphatics and nerves. The veins are then severed and closed off. The spermatic cord is sutured, and the incision is closed. Despite being one of the most common procedures for treatment of varicocele, there are several complications associated with the procedure. The three most significant complications include recurrent or persistent varicocele, hydrocele (e.g., collection of fluid around the testis) formation, and damage to the testicular artery.

In laparoscopic varicocele ligation procedures, a camera and several small instruments are introduced to the abdomen and the veins that comprise the varicocele are clipped. This procedure has shown poorer long-term success rates compared to other treatment methods. Additionally, the procedural complications of a laparoscopic varicocele ligation can be far more serious than other approaches. The occurrence rate of hydrocele after surgery is higher with this approach.

Interventional radiology procedures have lower complications and significantly faster recovery rates compared to surgical procedures for the treatment of varicocele. The procedure involves advancing a catheter into the spermatic vein through the renal vein. A microcatheter is navigated down the spermatic vein, as far as the inguinal canal, and the catheter is used to deliver sclerosing agents or embolization coils. Usually contrast is injected to verify occlusion and visualize any collateral blood vessels. If collateral blood vessels are observed, additional coils or sclerosant is placed to minimize recurrence. Usually multiple coils are placed along the length of the spermatic vein to completely occlude the spermatic vein. Hydrocele formation is not frequently observed with interventional radiology procedures for the treatment of varicocele.

Percutaneous sclerotherapy (or embolization) using interventional radiological techniques is effective in eliminating the pathophysiological hydrostatic pressure from faulty, malfunctioning one-way valves in the ISV and/or the accompanying network of retroperitoneal venous bypasses. Both techniques enable control and occlusion of the entire network of venous bypasses associated with the malfunctioning ISV on both sides that produce elevated hydrostatic pressure regardless of the diameter of the veins. Elimination of the pathological hydrostatic pressure by these treatments restores normal arterial oxygenated blood flow and normal supply of nutrient materials to the seminiferous tubules—the sperms production site. Drawbacks of using interventional radiological procedures include high recurrence rates. Another drawback of radiological intervention-based procedures is that they do not address the symptoms associated with nutcracker syndrome. High pressures in the renal vein could be the primary reason for dislodgement of embolization coils, migration of coils to lungs and high recurrence rates in varicocele and pelvic congestion.

Systems, apparatus, and methods described herein provide options for treating varicocele and its associated complications (e.g., diseases and/or medical conditions) without certain drawbacks of existing treatment options.

### Systems, Devices, and Methods for Creation of Fluid Connection Between Two or More Blood Vessels—Anastomosis-Based Devices for Treatment of Varicocele

Set forth below is a detailed description of various embodiments of a medical device and method for the creation of a fluid connection between two or more blood vessels to improve venous blood flow from the testes as well as a method for the delivery, positioning, and operation of the medical device. Establishing a fluid connection between the inferior region of testicular vein and a surrounding blood vessel equalizes the blood pressure with the connecting blood vessel.

There are several advantages to using a fluid connection to treat varicocele, including, for example: typically a single connection can be made for an entire testicular vein bed to equalize testicular venous blood pressure, including its collaterals; the approach can be used in patients with nutcracker syndrome or left renal vein compression, as it allows for an alternative pathway for renal venous blood drainage apart from renal veins; avoids the need for multiple embolization coils to ensure complete blockage of the spermatic vein; etc. The use of a fluid connection also reduces the exposure of the prostate to free testosterone compared to embolization or ligation of the testicular vein(s). The testicular veins are the primary route for drainage of testicular venous blood. Ligation or occlusion of the testicular vein(s) forces blood drainage through other smaller blood vessels into the iliac vein. Since the back pressure of the testicular venous blood following ligation or occlusion of the testicular vein(s) is reduced, the exposure of the prostate to testosterone-rich venous blood is reduced for a patient receiving this treatment. However, with ligation or occlusion, the prostate would still be exposed to higher-than-normal, supra-physiological levels of testosterone concentrations. Conversely, in accordance with embodiments described herein, creating a fluid connection can allow for drainage of the majority of testosterone-rich blood directly into the greater venous blood vessels, thereby reducing exposure of the prostate to testosterone-rich blood. The use of a fluid connection can also lead to immediate, long-standing pain relief, while inflammation of the spermatic cord and testicular vein(s) can reoccur post-occlusion or post-ligation as the testicular vein, and its collaterals, further degrade over time.

Systems, devices, and methods described herein configured for creating a fluid connection can include those that lead behind an implant (e.g., a fluid shunt) and those that create fluid connections without implants.

### 1. Approaches Using Implants

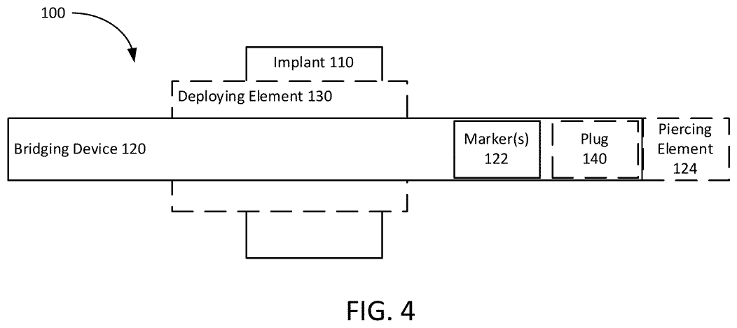

FIG. 4 schematically depicts an example device 100 for forming a fluid connection and deploying an implant (e.g., fluid shunt). The device 100 can include a bridging device 120. In some embodiments, the bridging device 120 can be configured to directly support an implant, e.g., in the case of a percutaneous bridging device supporting an implant. In some embodiments, the bridging device 120 can be implemented as guidewire or other guiding device, on which or within which a device and/or deploying element 130 (e.g., a catheter such as, for example, a balloon catheter) supporting an implant 110 can be guided to a target site.

In an embodiment, the implant 110 can be a fluid shunt. The device 100 can be configured for navigation to a target site and to deploy the implant 110 at the target site. The target site can be a pathway that extends between two vessels, e.g., a spermatic or ovarian vein where there is an occlusion or faulty valve to a neighboring vein (e.g., deep circumflex vein, femoral vein, epigastric vein, etc.). The bridging device 120 can be navigated to a target site using an endovascular approach (e.g., via one or more vessels), a percutaneous approach, a laparoscopic approach, etc.

The bridging device 120 can be a catheter, shaft, guidewire, etc. The bridging device 120 can include steering mechanisms, e.g., pull wires, compression coils, etc., for steering a distal tip of the device 120 through passages defined by patient anatomy and/or within a catheter (e.g., a catheter positioned endovascularly within a vessel of an individual). The bridging device 120 can be configured to have sufficient flexibility for navigating suitable anatomy, including anatomy near a spermatic vein and/or ovarian vein of a subject. In some embodiments, the bridging device 120 can have pre-shaped geometry or shapeable structure for facilitating introduction, navigation, and positioning of the bridging device 120.

The implant 110 can be used to create a fluid connection between two or more vessels such that improved blood flow can be achieved. The implant 110 can have a tubular or elongate structure. The implant 110 can have a flexible section (or be flexible throughout). The implant 110 can optionally include at least one end that includes an anchoring element for anchoring to a vein.

The device 100 can optionally include a deploying element 130, which can be configured to deploy the implant 110. In an embodiment, the deploying element 130 can be a balloon or basket that expands to deploy the implant 130. Alternatively, the deploying element 130 can be a pusher, a shaft, or some other structure that can be manipulated to deploy the implant 110. In some embodiments, the bridging device 120 may not include a deploying element, but the implant 110 can automatically release or decouple from the bridging device 120, e.g., in response to body heat that causes the implant 110 to expand, frictional forces between the implant 110 and surrounding anatomy (e.g., a vein wall), or other factors. In some embodiments, the deploying element 130 can be supported on the bridging device 120. Alternatively, the deploying element 130 can be supported on a separate device (e.g., a deployment device) that can be advanced along and/or positioned about the bridging device 120 to deploy the implant 110. For example, in an embodiment, the bridging device 120 can be a guidewire, and a deployment device implemented as a balloon catheter can be advanced over the guidewire to a target site such that a deploying element 130 implemented as a balloon can be used to deploy an implant 110.

As described above, in treating varicocele, the creating of a fluid connection between the spermatic vein can reduce or eliminate hydrostatic pressure, thereby improving venous circulation from the testes. The spermatic vein, however, crosses many of its neighboring veins at an angle. Given the small size of these blood vessels and the acute angle of incidence with respect to one another, there is a narrow surface area available at a cross-over point for the creation of a fluid connection. Accordingly, it is important to ensure accurate positioning of the bridging device. In some embodiments, the bridging device 120 can include one or more marker(s) 122 or other elements (e.g., extrusions, ridges, etc.) for visualization of the bridging device 120 during use. In some embodiments, the bridging device 120 can include visualization elements, e.g., a camera or lens, a light source, etc. for visualizing a position of the bridging device 120 during use.

In some embodiments, the device 100 can optionally include a plug 140. The plug 140 can be used to plug or close openings within a vessel (e.g., a vein) or tissue that do not form a part of a fluid connection between target vessels. For example, when using a percutaneous approach, a bridging device 120 may form openings in one or more vessels in addition to the openings that form part of the fluid connection between two vessels. The bridging device 120 can be equipped with a plug 140, which the bridging device 120 can deploy in the additional openings, e.g., to prevent complications such as a hemorrhage. In some embodiments, the bridging device 120 can include one or more components (e.g., shafts, push rods or sleeves, etc.) for deploying the plug 140. In some embodiments, the implant 110 can be include (or be shaped to function as) a plug. Alternatively or additionally, in some embodiments, the device 100 can include mechanisms for delivery of energy, suturing, delivery of hemostatic agents, etc. for preventing hemorrhaging.

In some embodiments, the bridging device 120 can optionally include a piercing element 124. The piercing element 124 can be a sharp end that is configured to pierce tissue. For example, when implemented as a percutaneous device, the bridging device 120 can include a shaft end that is configured to pierce through tissue to position a portion of the bridging device 120 between two vessels (e.g., the spermatic vein and a neighboring vein). When implemented as an endovascular device, the bridging device 120 can include a sharp end that is configured to form an opening in a vessel (e.g., the spermatic vein and a neighboring vein) to form the fluid connection between the vessel and a neighboring vessel.

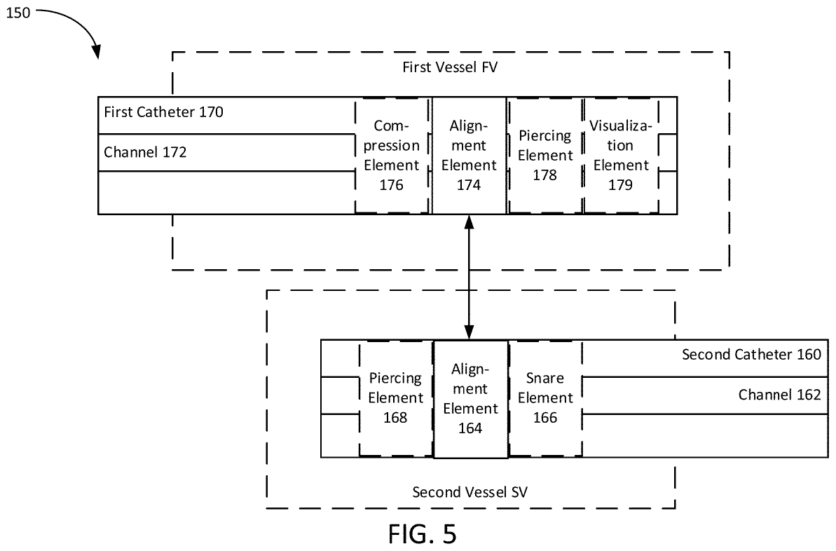

FIG. 5 depicts a system 150 for guiding a bridging device 120 to a target site, e.g., to create a bridge or fluid connection between two vessels. The system 150 can include a first catheter 170 and a second catheter 160. The first catheter 170 can be positioned within a first vessel FV, and the second catheter 160 can be positioned within a second vessel SV. In some instances, the first vessel FV can be a spermatic or ovarian vein, and the second vessel SV can be a neighboring vein to the spermatic or ovarian vein. It can be appreciated, however, that the first and second vessels can be any blood vessels within the human body, and that systems, devices, and methods described herein can be adapted for use with such vessels.

The first catheter 170 can include a channel 172, e.g., for introduction of a medical device. In embodiments described herein, the channel 172 of the first catheter 170 can be configured to receive a bridging device (e.g., bridging device 120), and be used to guide or direct the bridging device to a target site (e.g., a cross-over point between the first and second vessels). In some embodiments, the channel 172 can also be configured to receive other instruments into a subject's body, e.g., surgical devices, visualization devices (e.g., for visualizing the bridging device 120 and/or the procedure of forming a fluid connection), shafts or other elongate members for manipulating an implant and/or bridging device (e.g., implant 110 or bridging device 120). In some embodiments, the channel 172 can be configured to deliver a fluid such as an agent (e.g., a contrast agent), chemical, therapeutic substance, anesthetic, etc. to a target site.

Similar to the first catheter, the second catheter 160 can include a channel 162. The channel 162 can be structurally and/or functionally similar to the first channel 172. For example, the channel 162 can be configured to receive a bridging device (e.g., bridging device 120) and/or other medical devices. In use, the channels 162, 172 can function together to position a portion of a bridging device between the first and second vessels. For example, a bridging device (e.g., bridging device 120 can be configured to be advanced along one channel (e.g., channel 172) until it reaches a cross-over point between the first and second vessels, and then be advanced from that one channel to the other channel (e.g., channel 162). The two channels 162, 172 can therefore be used to support and precisely position a bridging device within patient anatomy. The bridging device, once positioned at a cross-over point between the first and second vessels, can be configured to deploy an implant (e.g., implant 110) to form a fluid connection between the first and second vessels.

As described above, when forming a fluid connection to treat varicocele (and its associated complications and medical conditions), the fluid connection oftentimes must extend between two small vessels at an acute angle of incidence with respect to one another. Therefore, the cross-over point between the vessels is small and therefore requires precise positioning of the bridging device and any catheters guiding the bridging device, such as, for example, catheters 160, 170. To allow for accurate positioning, alignment, and stabilization of the catheters 160, 170, each catheter 160, 170 can include one or more alignment element(s) 164, 174. The alignment element(s) 164, 174 can include, for example, magnets or electromagnets (e.g., neodymium magnets) that align the openings for extending a bridging device (e.g., bridging device 120) between the catheters 160, 170. Additionally or alternatively, the alignment element(s) 164, 174 can include mating structure or interlocking features (e.g., protrusions and/or curvatures) that facilitate alignment between the catheters 160, 170. In use, the catheters 160, 170 can be navigated within the first and second vessels, respectively, to the cross-over point between the first and second vessels and then aligned using the alignment element(s) 164, 174 before extending a bridging device (e.g., bridging device 120) between the two catheters 160, 170. The alignment of the two catheters 160, 170 can be automatic and/or require activation of electric energy.

In some embodiments, one or both of the catheters 160, 170 can optionally include a piercing element 168, 178. The piercing element(s) 168, 178 can be configured to pierce through tissue (e.g., a vessel wall). The openings formed by the piercing element(s) 168, 178 can be for extending a bridging device (e.g., bridging device 120) from one catheter 160, 170 to the other. The piercing element(s) 168, 178 can be implemented as a needle, a guidewire, or other structure including a sharp end for piercing through tissue.

In some embodiments, at least one of the catheters 160, 170 can optionally include a visualization element 179. The visualization element 179 can be used to visualize the creation of a fluid connection between the first and second vessels. The visualization element 179 can include, for example, lens, camera, light sources, channels for delivering fluid contrast agents, etc. While not depicted, the first and second catheters 160, 170 can include markers (e.g., radiopaque markers) or other indicia for facilitating confirmation of the placement of the catheters 160, 170 within the patient anatomy before extending a bridging device (e.g., bridging device 120) between the catheters 160, 170.

In some embodiments, at least one of the catheters 160, 170 optionally includes a snare element 166. For instance, the snare element 166 can be located on the second catheter 160 and be configured to catch or secure a bridging device (e.g., bridging device 120) to pull the bridging device into the second catheter 160. In such instance, the first catheter 170 can be configured to receive the bridging device such that the bridging device can be advanced to the cross-over point between the first and second vessels. The bridging device can then be advanced out of the first catheter 170 and be caught by the snare element 166. In some embodiments, at least one of the first or second catheters 160, 170 can optionally include a stopping element configured to stop advancement of the bridging device such that the bridging device is not over-extended into the second catheter 160 (e.g., a surface or edge that mates with a surface or edge on the bridging device to prevent further advancement, or a structure incorporated into the snare element 166 that prevents the snare element 166 from further advancing the bridging device).

In some embodiments, at least one of the catheters 160, 170 can optionally include a compression element 176. The compression element 176 can be configured to bring the two catheters 160, 170 in closer proximity to one another, e.g., to facilitate bridging of a bridging device across the two catheters.

One or more of the first and second catheters 160, 170, the bridging device 120, and the implant 110 can be bundled together as a kit. If a catheter requires navigation through a blood vessel with valves, a valvulotome can be provided as part of the kit. In some embodiments, the disposable components (e.g., first and second catheters 160, 170, guidewires, etc.) can be bundled together in a single package. Other components in such a package optionally can include one or more of: a syringe, Luer-Lock adapter fittings, access sheath, instructions for use, any wirings or cables needed to operate and use the catheters, sterilized fluids, glue, etc.

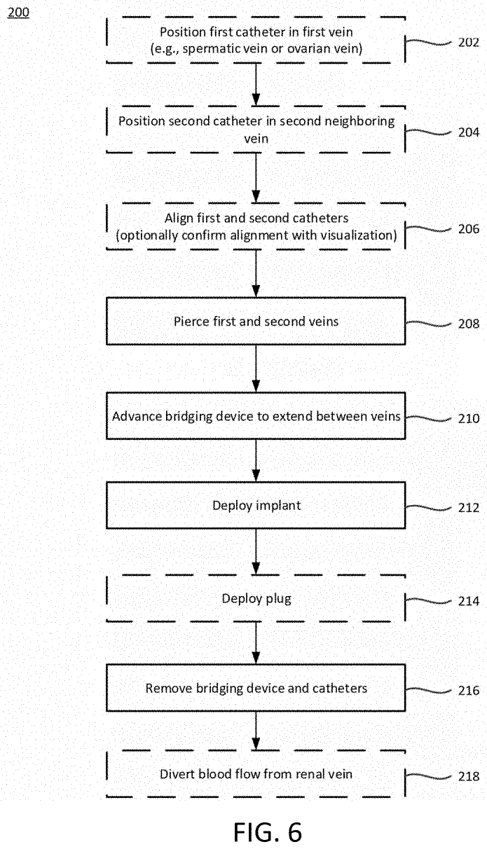

FIG. 6 depicts an example method 200 of forming a fluid connection between two blood vessels. In some instances, e.g., when forming the fluid connection using an endovascular approach or using catheters to guide a bridging device, a first catheter (e.g., first catheter 170) can optionally be positioned within a first vein (e.g., a spermatic or ovarian vein), at 202. And a second catheter (e.g., second catheter 160) can optionally be positioned within a second vein neighboring the first vein, at 204. The first and second catheters can be aligned with one another, e.g., using alignment elements (e.g., alignment elements 164, 174), at 206. Optionally, the alignment of the first and second catheters can be confirmed using visualization, markers, or other suitable methods. In other instances, e.g., when forming the fluid connection using a percutaneous approach, no first or second catheters may be used and therefore method 200 does not include 202-206.

At 208, the first and second veins can be pierced (e.g., using piercing elements 168, 178 on first and second catheters, or using a piercing element 124 on a bridging device). Once pierced, a bridging device (e.g., bridging device 120) can be extended from one of the first and second veins to the other of the first and second veins, at 210. In instances where the first and second catheters are used to guide the bridging device (e.g., when using an endovascular approach), the bridging device can be advanced through a channel (e.g., channel 162, 172) of one catheter into a channel (e.g., channel 162, 172) of the other catheter. In some embodiments, the catheter that receives the bridging device can include a snare element (e.g., snare element 166) that can catch the bridging device to guide or pull it into the second catheter. In instances where first and second catheters are not used to guide the bridging device (e.g., when using a percutaneous approach), the bridging device can be advanced on its own from one vein to the other vein. Throughout the process, one or more visualization elements or markers (e.g., cameras, lens, light sources, and/or radiopaque markers) can be used to confirm the advancement and alignment of the first and second catheters and to confirm the advancement and positioning of the bridging device.

When the bridging device is properly positioned, e.g., with a portion of the bridging device that supports an implant (e.g., implant 110) being positioned between the first and second veins, the implant can be deployed, at 212. The bridging device can then be removed, at 216, and optionally a plug can be deployed to seal one or more openings in the veins separate from the fluid connection, e.g., to prevent a hemorrhage, at 214. Alternatively or additionally, a hemorrhage can be prevented via delivery of an energy source, suturing, hemostatic agents, or any combination thereof. The first and second catheters (and any other devices used during the procedure) can also be removed, at 216.

In some embodiments, blood flow from the renal vein can also be diverted toward the target vessel and/or fluid connection, e.g., to increase a pressure difference across the implant, at 218. Increasing the pressure difference can improve a success rate of the fluid connection, e.g., by reducing a likelihood of undesirable clotting.

While systems, devices, and methods disclosed herein are described with reference to treating varicocele and related medical conditions and/or diseases, it can be appreciated that any such systems, devices, and methods can be applied to form fluid connections in other vessels within the body, including for example, other vessels within female and/or male reproductive systems, other vessels in the urinary-tract system, other vessels in the cardiovascular system, other vessels in the brain, other vessels in the legs, arms, or other locations within patient anatomy, etc.

### 1.1 Endovascular Devices: Fluid Shunt

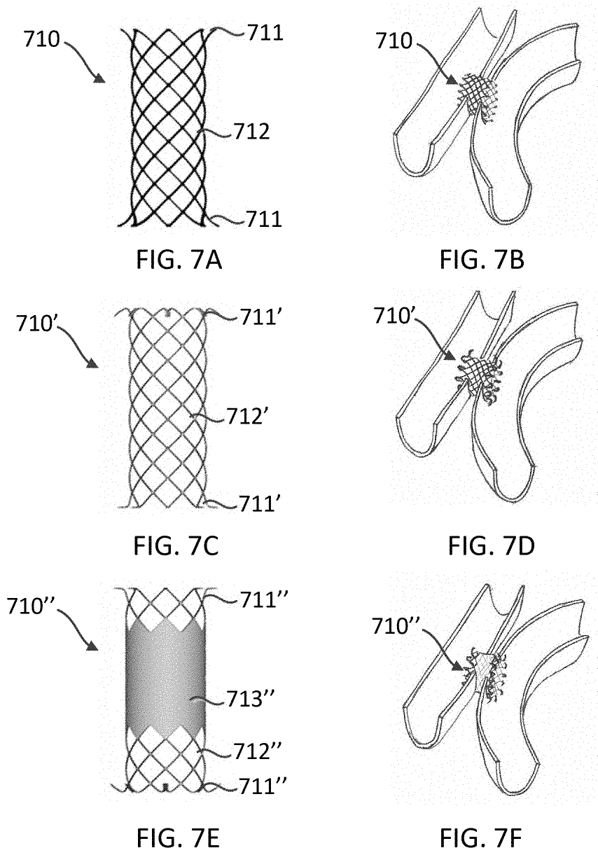

In some embodiments, an implant (e.g., implant 110) can be implemented as a fluid shunt. Fluid shunts 710, 710′, 710″, as shown in FIGS. 7A-7F, respectively, may be used to redirect venous blood from the testes to at least one other blood vessel to improve blood flow. In various possible embodiments, a fluid shunt may comprise several geometries including, but not limited to, an expandable stent, conduit, hollow tube, or channel. The fluid shunts 710, 710′, 710″ include an expandable structure to allow for introduction and navigation into the intended blood vessel. The fluid shunts 710, 710′, 710″ can include a fluid shunt body 712, 712′, 712″, which can be comprised of any suitable biocompatible material, including metals (such as nitinol and stainless steel), plastics, polymers, fabrics, grafts, biological tissue, or a combination thereof. In some embodiments, such as the fluid shunt 710″, a portion of the fluid shunt body 712″ may incorporate a material covering 713″ that may be comprised of any suitable biocompatible material, including, for example, fabrics, plastics, polymers, biological tissue, synthetic materials, or a combination thereof. The inner diameter of the fluid shunts 710, 710′, 710″ can be variable to allow adequate drainage of blood. Additionally, a variable inner diameter allows for effective anchoring of the fluid shunts 710, 710′, 710″ between two blood vessels of dissimilar diameters. The different fluid shunts 710, 710′, 710″ shown in FIGS. 7A, 7C and 7E have varying ends to connect to the blood vessels based on variable diameter or sizes and/or mechanical properties of the fluidic connection.

In an example embodiment, one end of a fluid shunt 710, 710′, 710″ can be positioned near the inferior-most aspect of the spermatic vein, near the inguinal canal ring. The other end of the fluid shunt 710, 710′, 710″ can be connected to a neighboring blood vessel in a manner that allows blood flow between the spermatic vein and the neighboring blood vessel. The neighboring blood vessel connected to the fluid shunt 710, 710′, 710″ may be the iliac vein, deep circumflex iliac vein, or any other suitable blood vessel. The fluid shunt 710, 710′, 710″ may be used to create a fluid connection between two or more veins so that improved blood flow from the testes is achieved. The ends of the fluid shunt 710, 710′, 710″ may have a closed configuration 711, 711′, 711″, or may comprise a shape enabling the fluid shunt 710, 710′, 710″ to anchor to at least one blood vessel (see FIGS. 7B, 7D, and 7F). In another embodiment, the fluid shunt 710, 710′, 710″ may comprise at least one terminal anchor (not depicted) connected by a collapsible or flexible member. The terminal anchor(s) may comprise any suitable rigid, biocompatible material including, for example, metals (such as nitinol and stainless steel), alloys, plastics, polymers, or a combination thereof. The flexible member can be comprised of another suitable biocompatible material including plastics, polymers, fabrics, grafts, biological tissue, or a combination thereof. In yet another embodiment, the fluid shunt 710, 710′, 710″ may comprise sharp ends 711, 711′, or 711″ on one side or both sides of the flexible member 712, 712′ or 712″ that pierce and/or anchor the fluid shunt 710, 710′, 710″ to the blood vessel wall as well as secure the two blood vessels close to each other.

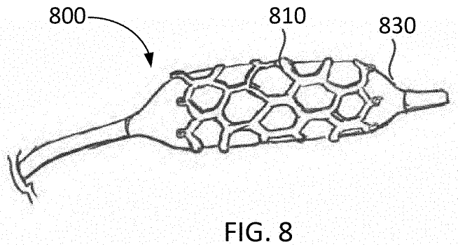

A fluid shunt (or other implant) may be delivered to the target location using several different mechanisms. In one particular embodiment, a fluid shunt is delivered and expanded via an inflatable balloon at the distal aspect of a balloon catheter 800, depicted in FIG. 8. The balloon catheter 800 can extend from a first vein (e.g., spermatic vein) to a second vein, and be an example of a bridging device (e.g., bridging device 120).

In one embodiment, the balloon catheter 800 comprises a structure with the appropriate material properties (e.g., lubricity, flexibility, torquability, column strength, bending tolerance, etc.) to navigate the tortuosity of the vascular system to reach the inferior-most aspect of the spermatic vein. In another embodiment, the balloon catheter 800 comprises a structure with material properties that vary along the length of the catheter to, for example, exhibit higher stiffness and/or lower flexibility proximal to the balloon and high flexibility and/or lower stiffness distal to the balloon. The balloon catheter 800 may be advanced over a support guidewire or a needle. The balloon catheter 800 comprises a hollow lumen allowing for the insertion of a guidewire, but can also comprise a rapid-exchange catheter tip. The balloon catheter 800 may also comprise one or more mechanisms allowing for catheter steerability in at least one direction. Steerability may be achieved via one or more push-pull members extending from the handle of the catheter to the distal tip of the catheter. Pushing and pulling on members facilitates deflection of the catheter tip in one or more directions. A balloon catheter 800 comprising steerability mechanisms may or may not require navigation over a guidewire to reach the target location in the spermatic vein. Once the balloon catheter 800, has been advanced over the guidewire and properly positioned between the target two blood vessels, an inflatable balloon 830 (e.g., a deployment element) at the end of the catheter is inflated to expand and deploy a fluid shunt 810 to create a fluid connection between the blood vessels.

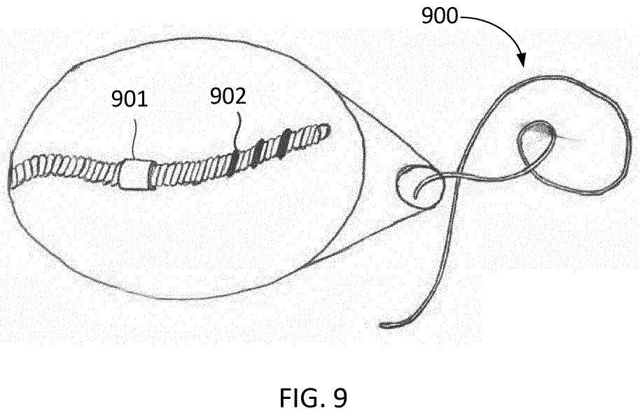

FIG. 9 depicts a bridging guidewire 900, which can also form a part of or be an example bridging device. The bridging guidewire 900 can be configured to cross over from the spermatic vein to a neighboring blood vessel. The bridging guidewire 900 may be visualized and navigated through the vasculature via several imaging modalities including, but not limited to, fluoroscopy, magnetic resonance imaging, computed tomography, ultrasound, doppler imaging, optical imaging, or a combination of one or more of these modalities. The bridging guidewire 900 may be comprised of any suitable biocompatible material, including metal, alloy, plastic, polymer or any combination thereof. The bridging guidewire 900 may comprise elements for visualization and/or facilitating the bridging between two vessels 901 and 902 via the aforementioned imaging modalities including grooves, radiopaque markers, extrusions, ridges, or any combination thereof. The bridging guidewire 900 may comprise a pre-shaped geometry or suitably shapeable materials including nitinol for improved introduction, navigation, and positioning within the body.

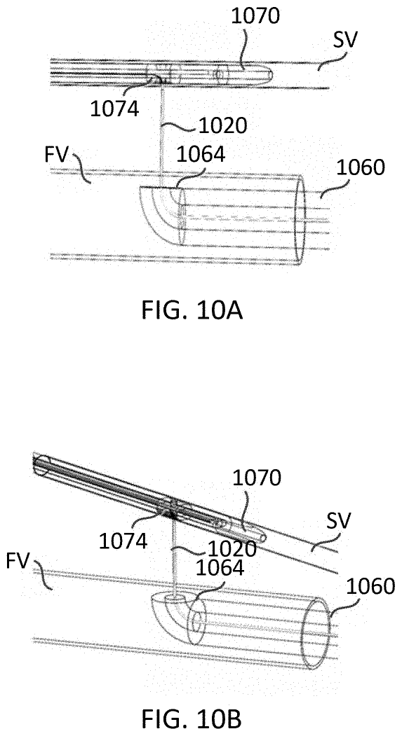

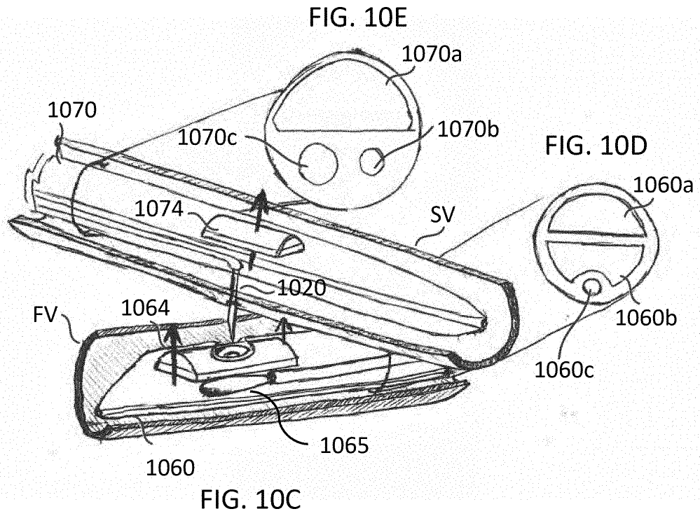

There are several methods and devices that can be used to guide a bridging guidewire (e.g., bridging guidewire 900) to create a bridge between two or more blood vessels. In one embodiment shown in FIGS. 10A-10C, a deployment catheter 1070 (e.g., a first catheter) is introduced into the vasculature and navigated to the spermatic vein SV and positioned in-line with the iliac vein or the femoral vein FV. In particular, the catheter can be advanced through the femoral vein FV and stopped when it advances to the iliac vein.

A receiving catheter 1060 (e.g., a second catheter) is introduced into the femoral vein FV and positioned in-line with the deployment catheter 1060. Conversely, the receiving catheter 1060 and the deployment catheter 1070 may be introduced into the spermatic vein and the femoral vein, respectively.

In some embodiments, a bridging guidewire can comprise one or more mechanisms allowing for steerability in at least one direction. Steerability may be achieved via one or more push-pull members (not shown) extending from the handle of the guidewire to the distal tip of the guidewire (see FIG. 10C).

The receiving catheter 1060 and/or the deployment catheter 1070 may comprise a mechanism for indicating, visualizing, verifying, and/or facilitating correct positioning and alignment of the catheters relative to each other, and may comprise one or more magnets, radiopaque markers, position sensors, or any combination thereof. In one embodiment, alignment of the receiving catheter 1060 and the deployment catheter 1070 may be achieved under fluoroscopy via radiopaque markers indicating the position where the two catheters should cross over. Once the catheters 1060, 1070 are properly positioned, oriented, and/or aligned with each other, the catheters may comprise element(s) to facilitate bringing the catheters into closer proximity. In one embodiment, the element(s) for facilitating the alignment of the catheters 1060, 1070 comprises two or more locking magnets or electromagnets, 1064, 1074. The locking magnets, 1064, 1074, may comprise high-strength neodymium magnets and/or interlocking features to secure the deployment catheter 1070 to the receiving catheter 1060. When the catheters have been locked or secured together, a piercing member may be advanced through the deployment catheter 1070 to pierce through the wall of encompassing blood vessel. The piercing member may comprise a guidewire 1020 (e.g., a bridging guidewire), a needle, and/or any other structure with the appropriate material properties for minimally-traumatic advancement of the piercing member through the walls of the blood vessels through which a fluid connection is being created.

In one embodiment, the piercing member is advanced through the deployment catheter 1070 and pierces through the spermatic vein at the crossover point. The advancement of the piercing member may be achieved through manual force alone or further enabled via radiofrequency ablation, plasma, or any other suitable means. The receiving catheter 1060 may comprise a catching mechanism 1065 (e.g., snare element) (see FIG. 10C) to catch the piercing member (e.g., bridging guidewire 1020). The receiving catheter 1060 may also comprise a hard-stop mechanism (not depicted) that prevents the piercing member from over-advancement beyond the receiving catheter.

In one embodiment, the bridging guidewire 1020 is advanced through the wall of the spermatic vein SV followed by advancement through the wall of the femoral vein FV and into a receiving mechanism on the receiving catheter 1060. Following the advancement of the bridging guidewire 1020 from the deployment catheter 1070 into the receiving catheter 1060, a balloon catheter (e.g., balloon catheter 800) can be advanced over the bridging guidewire 1020. Once the balloon catheter has been properly positioned over the bridging guidewire 1020 and the inflatable balloon at the distal aspect of the catheter is inflated to deploy a fluid shunt (e.g., an implant, or fluid shunts 710, 710′, 710″) and create a fluid connection between the blood vessels. Following positioning, expansion, and securing the fluid shunt in place between the femoral vein and the spermatic vein, the catheters, guidewire, etc. are then removed. The process may be repeated bilaterally if needed.

FIGS. 10D and 10E depict cross-sectional views of the receiving catheter 1060 and the deployment catheter 1070, respectively. As depicted, each catheter 1060, 1070 can include a plurality of lumens or channels for receiving different components of the system. For example, the deployment catheter 1070 can include a first lumen 1070a for placement of alignment member, 1074, a second lumen 1070b for guidewire advancement and a third lumen 1070c for a bridging guidewire. Similarly, the receiving catheter can include a first lumen 1060a for a second alignment member, a second lumen 1060b for a catching mechanism, and a third lumen 1060c for catheter advancement over a guidewire.

### 1.2 Percutaneous Devices: Fluid Shunt

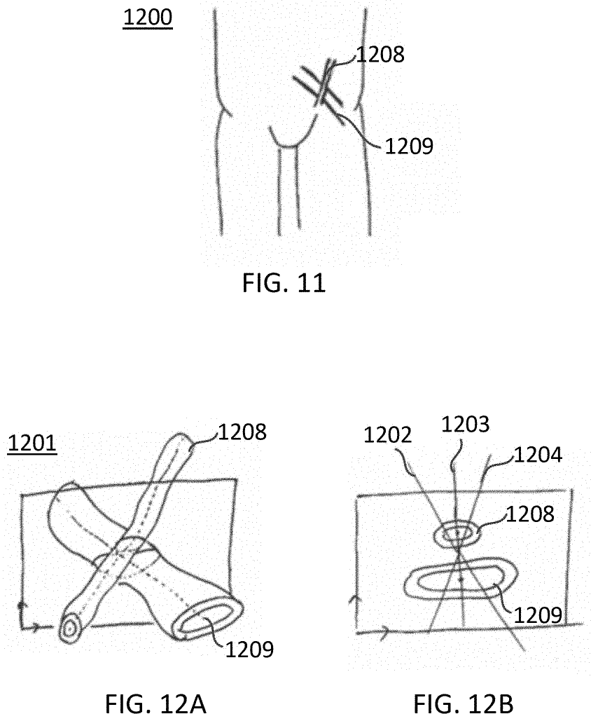

In another example method, the introduction, placement, and implantation of the bridging member is achieved via a percutaneous approach using imaging guidance. As an illustrative example, the percutaneous deployment of the bridging member is summarized by the graphical sequence summarized in FIGS. 11-15B.

As illustrated in FIGS. 11-15B, an example percutaneous approach for the creation of a fluid connection between two blood vessels with a fluid shunt (e.g., an implant) can include the following steps.

First, based on patient-specific anatomical data, a target pathway through the body is determined, as depicted in FIGS. 11-13. The anatomical data for the patient may be obtained using, but not limited to, ultrasound, magnetic resonance imaging, computer tomography, doppler, optical imaging, fluoroscopy, radiography, or any combination thereof (see FIG. 11). The determined pathway insects with, at least in part, a first blood vessel 1208 and a second blood vessel 1209, of which at least one blood vessel comprises the spermatic vein. There are multiple possible, adequate, and/or clinically relevant pathways that the determined pathway may comprise, e.g., pathways 1202, 1203, 1204. An example trajectory may intersect with the two target blood vessels while avoiding any critical anatomical structures.

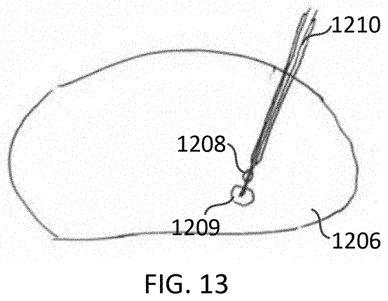

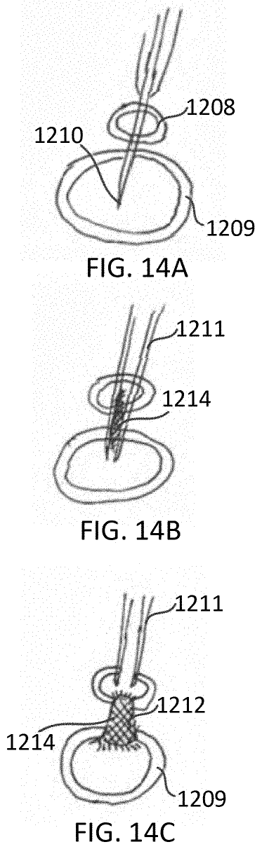

A bridging member 1210 (e.g., bridging device 120), which may comprise any suitable structure including, but not limited to, a needle, a probe, a catheter, or a minimally invasive delivery tool, is advanced along the determined pathway, as depicted in FIGS. 13 and 14A. The bridging member may comprise position sensors that determine the position of the bridging member in three-dimensional space in real-time. The advancement of the bridging member may be guided in real-time based on the patient's anatomical data and the position of the bridging member in three-dimensional space with respect to reference markers. The reference markers may comprise anatomical structures, fiducial markers, bony structures, or any other suitable marker. The advancement of the bridging member may be performed by a robotically-assisted device or system.

Once the bridging member is in the desired location, a shunt-deploying member (or deployment device) 1211 may be advanced over the bridging member 1210, as depicted in FIG. 14B. The shunt-deploying member 1211 may comprise metal or alloy tubing for higher column strength, and may additionally comprise a balloon catheter.

When the shunt-deploying member is in the desired location, the fluid shunt 1214 is deployed to create a fluid connection 1212 between the target blood vessels, as depicted in FIG. 14C. In some embodiments, the shunt-deploying member 1211 can include a deploying element (e.g., a deploying element 130) for deploying the fluid shunt 1214. As an illustrative example, a deploying element implemented as a balloon may be inflated to expand and deploy the shunt in place. The balloon is then deflated and retracted, leaving the shunt anchored in place.

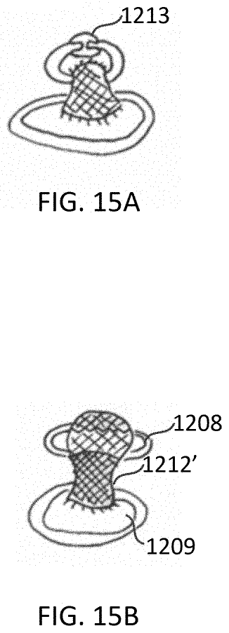

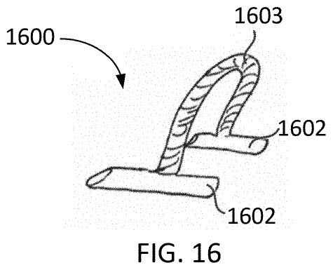

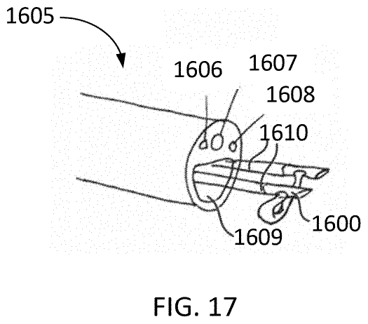

The bridging member may optionally include a mechanism for preventing hemorrhage of at least one target blood vessel prior to complete removal of the bridging member. The mechanism for preventing hemorrhage may comprise deploying a blood vessel plug 1213 (e.g., as depicted in FIG. 15A), delivery of an energy source, suturing, hemostatic agents, or any combination thereof. Alternatively, at least one of the target blood vessels may be closed using external pressure or a pre-shaped shunt 1212′ that extends through the lumen of the first blood vessel, as depicted in FIG. 15B.

### 1.3 Laparoscopic Devices: Fluid Shunt

In another embodiment, a bypass shunt 1600 is used to redirect venous blood from the testes to at least one other blood vessel to improve blood flow. In one particular embodiment, one end of the bypass shunt 1600 is positioned near the inferior-most aspect of the spermatic vein, near the inguinal canal ring. The other end of the bypass shunt 1600 is connected to a neighboring blood vessel in a manner that allows blood flow between the spermatic vein and the neighboring blood vessel. The neighboring blood vessel connected to the bypass shunt 1600 may be the iliac vein, deep circumflex iliac vein, or any other suitable blood vessel. The bypass shunt 1600 may be used to create a fluid connection between two or more veins so that improved blood flow from the testes is achieved. The ends of the bypass shunt 1600 may comprise a shape enabling the device to anchor to at least one blood vessel. The bypass shunt 1600 may comprise at least one terminal anchor connected by a collapsible or flexible member 1601. The terminal anchor(s) may comprise any suitable rigid, biocompatible material including, for example, metals (such as nitinol and stainless steel), alloys, plastics, polymers, or a combination thereof. The flexible member can be comprised of a suitable biocompatible material including, for example, plastics, polymers, fabrics, grafts, biological tissue, or a combination thereof. In yet another embodiment, the bypass shunt 1600 may comprise sharp ends that pierce and anchor the device to the blood vessel wall as well as secure the two blood vessels close to each other.

In one example embodiment, the bypass shunt 1600 comprises two piercing members, 1602 and a bypass member 1603. Together, these features allow for bidirectional blood flow through the bypass member 1603. The piercing members 1602 are configured to pierce through the wall of target vessels with minimal trauma and bleeding. Furthermore, the piercing members are configured to anchor into the blood vessel following puncture. The diameter of the piercing members may range from, but is not limited to about 1 to about 15 mm. The piercing members 1602 may comprise any suitable biocompatible material, including, for example, metal, alloy, coated metal, polymers, plastic, coated plastic, or any combination thereof. The piercing members 1602 may comprise hollow tubing or a part thereof. The piercing members 1602 may comprise curved surfaces to stay flush with the blood vessel walls, or may alternatively comprise a suitably flexible material to conform to the curvature of the surrounding anatomy.

The bypass member 1603 may comprise a flexible and kink-resistant material configured to allow for continued blood flow through the bypass shunt 1600. The bypass member 1603 may comprise any suitable biocompatible material with sufficient flexibility including, but not limited to, fabrics, polymers, biological tissue, graft, plastics, metals, or any combination thereof. The bypass shunt 1600 may be placed under direct visualization via laparoscopic, endoscopic, or any other minimally invasive approach. The bypass shunt 1600 may be placed using the shunt delivery system 1605. Alternatively, a Natural Orifice TransEndoluminal Surgery (NOTES) approach may be used to deliver the bypass shunt. NOTES employs the use of a natural orifice to get as close as possible to a target area without requiring the need for an incision, then an internal incision is made to gain access the surgical workspace. Following the completion of the procedure, the internal incision is closed, allowing for quicker recovery time and minimal scarring.

The shunt delivery system 1605 may comprise a rigid trocar-like instrument configured for deployment via a single incision. The shunt delivery system 1605 may be rigid or flexible, and may comprise any suitable biocompatible material including, but not limited to, metal, alloy, plastic, polymer, silicone, or any combination thereof. In one particular embodiment, the shunt delivery system 1605 comprises an optical module 1606, an illumination module 1607, a lens cleaning channel 1608 that can be used to create and maintain pneumoperitoneum as well as clean the optical module 1606, and a working channel 1609. The working channel 1609 may be used to deliver the bypass shunt 1600. In one particular embodiment, the two piercing members 1602 of the bypass shunt 1600 are connected to delivery members 1610 of the shunt delivery system 1605. The delivery members 1610 may be advanced and retracted from within the working channel by any suitable means including, but not limited to, mechanical and electrical actuators, manual advancement, pneumatics, hydraulics, or any combination thereof. The working channel 1609 has a sufficient internal diameter to allow for introduction, delivery, and placement of the bypass shunt 1600.

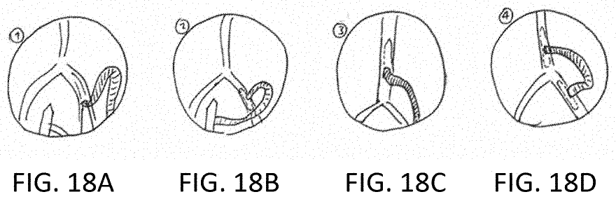

An example method for the deployment and implantation of the bypass shunt 1600 is summarized by the graphical sequence as depicted in FIGS. 18A-18D, and includes the following steps.

First, one of the piercing members 1602 is advanced into a target vessel. Example target blood vessels include, but are not limited to, the spermatic vein, epigastric vein, iliac vein, ovarian vein, and other venous and arterial blood vessels. The first piercing member 1602 is advanced through the vessel wall until the piercing member is securely positioned within the vessel. In one example embodiment, the piercing member 1602 comprises an elongated shape with a length that measures greater than its width or diameter. The elongated shape of piercing member 1602 functions to resist removal from the blood vessel after puncture, however, other the bypass shunt 1600 may comprise other means for resisting removal including an adhesive coating, an externally applied adhesive, and creation of a hemostatic bond between the vessel and bypass shunt via the delivery of radiofrequency energy.

Second, the first piercing member 1602 is decoupled from the delivery member of the shunt delivery system 1605 if used.

Third, the second piercing member 1602 is advanced and secured into another target vessel similarly to the first piercing member. The bypass member 1603 is in fluid-communication with the first and second piercing members and allows for blood flow between the target vessels.

### 1.4 Combination of Laparoscopic and Endovascular Devices: Fluid Shunt

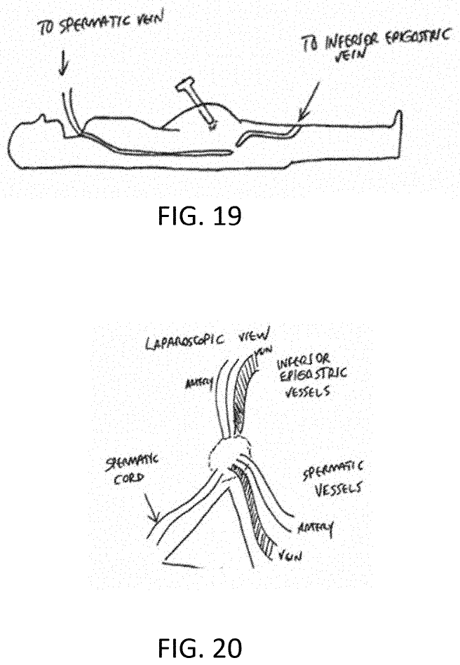

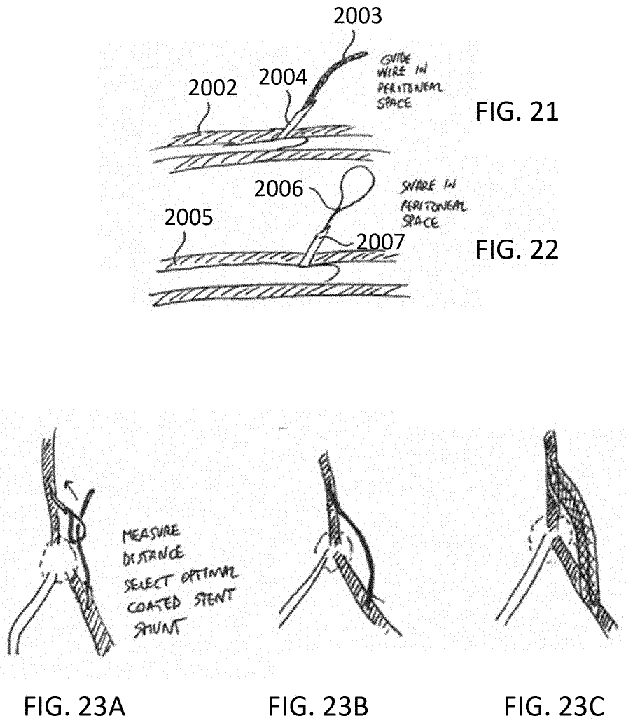

In accordance with another example embodiment, there are several methods and devices that can be used to guide a bridging member 2003 (e.g., a bridging device or a portion of a bridging device) to create a bridge between two or more blood vessels. In an example embodiment shown in FIGS. 19-23C, a snare-deploying catheter 2005 is introduced into a target blood vessel, and a bridge-deploying catheter 2002 is introduced into another target blood vessel. A bridging member 2003 and a snare member 2006 are configured for deployment through the wall of at least two target blood vessels for the formation of a fluid connection.

As depicted, a bridge-deploying catheter 2002 (e.g., a first catheter) is introduced into a first target blood vessel. A snare-deploying catheter 2005 (e.g., a second catheter) is introduced into a second target blood vessel and positioned in close-proximity with the bridge-deploying catheter. As an illustrative example, the bridge-deploying catheter 2002 and the snare-deploying catheter 2005 may be introduced into the spermatic vein and the femoral vein (or vice versa), respectively. The bridge-deploying catheter 2002 and the snare-deploying catheter 2005 may comprise a means for indicating, visualizing, verifying, and/or facilitating correct positioning and alignment of the catheters relative to each other, and may comprise one or more magnets, radiopaque markers, position sensors, or any combination thereof. In one embodiment, positioning of the bridge-deploying catheter 2002 and the snare-deploying catheter 2005 may be achieved under fluoroscopy via radiopaque markers indicating the proximity of the two catheters. Once the bridge-deploying catheter 2002 and the snare-deploying catheter 2005 are properly positioned, oriented, and/or aligned with each other, the catheters may comprise a means to facilitate bringing the catheters into closer proximity. In one embodiment, the means for facilitating the alignment of the bridge-deploying catheter 2002 and the snare-deploying catheter 2005 comprises two or more locking magnets or electromagnets. The locking magnets may comprise high-strength neodymium magnets and/or interlocking features.

When the catheters have been properly positioned, a piercing member on both the bridge-deploying catheter 2002 and the snare-deploying catheter 2005 is advanced through the wall of the encompassing blood vessels. The piercing member may comprise a guidewire, a needle, and/or any other structure with the appropriate material properties for minimally-traumatic advancement of the piercing member through a blood vessel. The piercing member may also comprise a connection to an energy source. The advancement of the piercing member may be achieved through manual force alone or further enabled via radiofrequency ablation, plasma, or any other suitable means.

The snare-deploying catheter 2005 comprises a catching mechanism in the form of a snare member 2006 configured to “catch”, or secure, a bridging member 2003 to allow for the advancement and placement of a fluid shunt over the bridging member. The snare-deploying catheter 2005 may also comprise a hard-stop mechanism (e.g., a stopping element) that prevents over-advancement of the bridging member. The bridging member 2003 is advanced through the bridge-deploying catheter 2002 into the snare member of the snare-deploying catheter. In one embodiment, a balloon catheter is advanced over the bridging member. Once the balloon catheter has been properly positioned over the bridging guidewire, an inflatable balloon at the distal aspect of the catheter is inflated to deploy a fluid shunt and create a fluid connection between the blood vessels. Following positioning, expansion, and securing the fluid shunt in place between the target blood vessels, all other devices are then removed. The process may be repeated bilaterally if needed.

In another embodiment, the bridging member 2003, bridge-deploying catheter 2002, and/or the snare-deploying catheter 2005 may also comprise one or more mechanisms allowing for steerability in at least one direction. Steerability may be achieved via one or more push-pull members extending from the handle of the guidewire to the distal tip of the catheter.

An example method for the deployment and implantation of a fluid shunt is summarized by the graphical sequence as depicted in FIGS. 23A-23C and comprises the following steps.

First, introduce one catheter (e.g., the bridge-deploying catheter 602 or the snare-deploying catheter 605) in the femoral or epigastric vein up to the inguinal ring. Second, introduce the other catheter in the spermatic vein down to the Triangle of Doom. Third, advance both the piercing members of both the bridge-deploying catheter 602 and the snare-deploying catheter 605 through the wall of the respective blood vessel either catheter resides in. Fourth, advance the snare member and the bridging member through the bridge-deploying catheter 602 and the snare-deploying catheter 605. Fifth, pass the bridging member through the loop of the snare member using any suitable means including, but not limited to, a laparoscopic grasper, magnetic guidance, or any other suitable means. Sixth, use the snare member to pull the bridging member guide wire inside the snare-deploying catheter. Seventh, advance a fluid shunt and its delivery system over the bridging members across the target vessels and deploy the shunt to create a fluid connection. Eighth, remove any instrumentation and close any incisions.

2. Approaches without Implants—Creation of Fistulas

In another example method for the treatment of, at least partially, varicocele, erectile dysfunction, infertility, nutcracker syndrome, BPH, bladder cancer, prostate cancer, pelvic congestion, ovarian cancer, polycystic ovarian syndrome, uterine fibroids, endometriosis, and/or hormonal disorders, the creation of a fistula between the spermatic vein(s) and another blood vessel can reduce or eliminate hydrostatic pressure, thereby improving venous circulation from the testes. Spermatic vein cross multiple blood vessels at an angle. Some of the target vessels may include, but not limited to, deep circumflex vein, femoral vein, epigastric vein. Given the small size of these blood vessels and the acute angle of incidence with respect to one another, there is a narrow surface area available at the crossover point, for creation of fistula. Therefore, it is important to have mechanisms for aligning and stabilizing the devices.

The access point for these devices can be through a percutaneous puncture or a laparoscopic access port. If though a laparoscopic access port, the device proximal handle can allow for navigation into the vasculature through the peritoneal cavity. If through a percutaneous access point, the device will be insertable through an incision on the skin and navigated directly to the vascular access site. Furthermore, any catheter based device may require a guidewire lumen for ease of navigation and can be replaced with a rapid exchange guidewire lumen for preservation of space in the distal end assembly.

As an illustrative example, a fistula forming device comprises at least one catheter introduced into at least one blood vessel. In one particular embodiment, a first fistula forming device, such as a source catheter (e.g., a first catheter), is introduced into the spermatic vein and a second fistula forming device, such as a target catheter (e.g., a second catheter), is introduced into the femoral vein. At least one fistula forming device may further comprise a recessed portion configured for accommodation of at least a portion of the other fistula forming device. At least one fistula forming device may further comprise at least one alignment member configured for visualization via fluoroscopy, ultrasound, optical imaging, electromagnetic imaging, or any combination thereof. The alignment member(s) may comprise at least one magnet and/or radiopaque marker indicating the position for which the first and second fistula forming devices should preferably cross over. Once the first and second fistula forming devices have been properly positioned and/or aligned within the target blood vessels, the first and second fistula forming devices may further comprise a mechanism to secure the positioning and/or alignment of the first and second fistula forming devices relative to each other. The aforementioned mechanism may comprise at least one magnet or any other suitable means.

After the first and second fistula forming devices have been secured in place, at least one fistula forming device is positioned within the recessed portion of another fistula forming device such that at least a portion of the spermatic vein is in close-contact with at least a portion of the femoral vein. At least one fistula forming device further comprises a compression element to induce compression, or pinching, of the target blood vessels together. A fluid connection consisting of a fistula between the spermatic vein and femoral vein, or any other suitable blood vessel, is created using any suitable mechanism including, but not limited to, the delivery of an energy source such as radiofrequency energy, plasma, heat, or any combination thereof. The mechanism for creating a fistula may also comprise a mechanical cutting mechanism such as a circular punch. After the creation of a fistula between the spermatic vein and another blood vessel (such as the femoral vein) all fistula forming devices are removed. The above process may be similarly performed on the other spermatic vein.

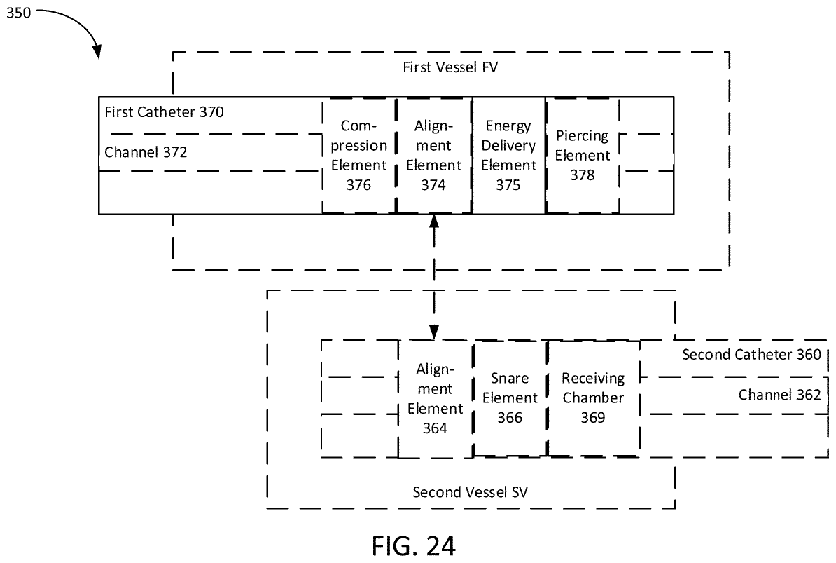

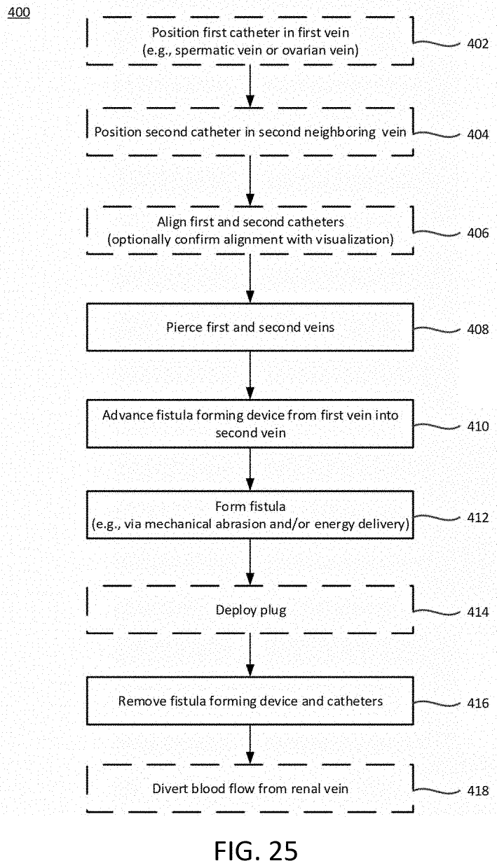

FIG. 24 schematically depicts an example system 350 for forming a fistula between two blood vessels. In some embodiments, the system 350 can include a first catheter 370 (e.g., a source catheter) and a second catheter 360 (e.g., a target catheter), e.g., which can navigate via vasculature to cross-over point between the two vessels. In other embodiments, the system 350 can include a single catheter, e.g., which is configured to form a fistula between the two vessels via a percutaneous approach. The first and second catheters 360, 370 can include component(s) that are structurally and/or functionally similar to the first and second catheters 160, 170, including components that are not specifically depicted in FIG. 24 (e.g., a visualization element).

The first catheter 370 can be positioned in or inserted into a first vessel FV, e.g., a spermatic or ovarian vein, or another vessel within patient anatomy. The second catheter 360 can be positioned in or inserted into a second vessel SV, e.g., a neighboring vein of the spermatic or ovarian vein, or other vessel within patient anatomy neighboring the first vessel FV.

The first catheter 370 can optionally include a channel 372, e.g., for receiving a medical device. In some embodiments, the channel 372 can be configured to receive a guidewire, e.g., for guiding the first catheter 370 to a target site (e.g., a cross-over point between the first and second vessels). In some embodiments, the channel 372 can be configured to receive a fistula-forming device, such as an energy delivery device. The fistula-forming device can be, for example, a guidewire or other flexible shaft with one or more energy delivery components (e.g., electrodes) for delivering ablation to form a fistula between the first and second vessels. In some embodiments, the channel 372 can also be configured to receive other instruments into a subject's body, e.g., surgical devices, visualization devices (e.g., for visualizing the distal end of the first catheter 370, the target site, and/or the procedure of forming a fistula), etc. In some embodiments, the channel 372 can be configured to deliver a fluid such as an agent (e.g., a contrast agent), chemical, therapeutic substance, anesthetic, etc. to a target site. In some embodiments, the channel 372 can be an optic channel, e.g., for delivery energy to a tip or distal end of the first catheter 370 to form the fistula.

The first catheter can include an energy delivery element 375 or other fistula-forming element. In some embodiments, the energy delivery element 375 can be, for example, one or more electrodes or conducting elements for delivering energy to surrounding tissue. The energy can include, for example, thermal, electrical, radiofrequency (RF), direct current (DC), cryogenic, chemical, or a combination thereof. In some embodiments, the energy delivery element 375 can be a window that allows energy delivered via an optic channel to be applied to surrounding tissue. In some embodiments, the energy delivery element 375 can be configured to change in configuration (e.g., deform, expand, etc.), e.g., to increase or decrease an area being ablated to form a fistula and/or to direct energy at a specific tissue area.

The first catheter 370 can optionally include a piercing element 378. The piercing element 378 can be configured to pierce through tissue (e.g., a vessel wall). The piercing element 378 can be configured to form openings in the vessel wall that allow an energy delivery device (e.g., a fistula forming device that is advanced through the first catheter 370, such as a guidewire) or energy delivery element (e.g., energy delivery element 375) to be positioned through the cross-over site between the first and second vessels. While not depicted, the second catheter 360, described below, can also include a piercing element for piercing through tissue, e.g., for deploying a snare element (e.g., snare element 366) and/or enabling the energy delivery device or element to be received in the second catheter 370.

In embodiments where first and second catheters 360, 370 are used together to form a fistula, it can be important to align and stabilize the two catheters 360, 370. As described above, the cross-over point between a spermatic vein and a neighboring vein can be at an acute angle and have a narrow surface area. As such, positioning and alignment of the first and second catheters 360, 370 can be important to ensure that a fistula is formed between the two vessels. To allow for accurate positioning, alignment, and stabilization of the catheters 360, 370, each catheter 360, 370 can include one or more alignment element(s) 364, 374. The alignment element(s) 364, 374 can include, for example, magnets or electromagnets (e.g., neodymium magnets). Additionally or alternatively, the alignment element(s) 364, 374 can include mating structure or interlocking features (e.g., protrusions and/or curvatures) that facilitate alignment between the catheters 360, 370. In use, the catheters 360, 370 can be navigated within the first and second vessels, respectively, to the cross-over point between the first and second vessels and then aligned using the alignment element(s) 364, 374 before forming a fistula (e.g., by advancing and/or activating energy delivery element 375).

In some embodiments, at least one of the catheters 360, 370 can optionally include a compression element 376. The compression element 376 can be configured to bring the two catheters 360, 370 in closer proximity to one another, e.g., to facilitate formation of a fistula between the first and second vessels.

When used with the first catheter 370, the second catheter 360 can be configured to stabilize the first catheter (e.g., using alignment elements 364, 374) and/or to receive a portion of the first catheter 370. For example, the first catheter 370 can include a component (e.g., energy delivery element 375 or a structure (e.g., shaft, platform, etc.) supporting energy delivery element 375) that is advanced through the cross-over point between the first and second vessels. The second catheter 360 can optionally include a snare element 366 for catching a portion of that component of the first catheter 370 and/or a receiving chamber 369 for receiving a portion of that component of the first catheter 370. The snare element 366 can be functionally and/or structurally similar to the snare element 166 described with reference to FIG. 5. The receiving chamber 369 can be an opening and/or a channel that can receive a portion of the first catheter 370 that is advanced through the cross-over point between the first and second vessels. In some embodiments, a structure (e.g., shaft, spline, guidewire, etc.) supporting the energy delivery element 375 can be advanced through the cross-over point and received in the second catheter 360 before energy is applied to form the fistula. Alternatively, a structure (e.g., a shaft, spline, guidewire, basket, platform) supporting the energy delivery element 375 can be advance through the cross-over point while delivering energy to surrounding tissue to form the fistula.

The first and second catheters 360, 370 can be bundled together as a kit. If a catheter requires navigation through a blood vessel with valves, a valvulotome can be provided as part of the kit. In some embodiments, the disposable components (e.g., first and second catheters 360, 370, guidewires, etc.) can be bundled together in a single package. Other components in such a package optionally can include one or more of: a syringe, Luer-Lock adapter fittings, access sheath, instructions for use, any wirings or cables needed to operate and use the catheters, sterilized fluids, glue, etc.

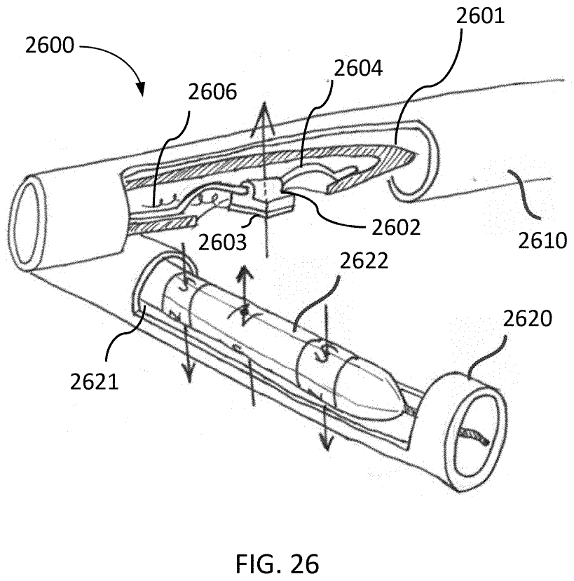

FIG. 26 depicts an example method 400 for forming a fistula between two blood vessels. A first catheter (e.g., first catheter 370) can optionally be positioned within a first vein (e.g., a spermatic or ovarian vein), at 402. And a second catheter (e.g., second catheter 360) can optionally be positioned within a second vein neighboring the first vein, at 404. The first and second catheters can be aligned with one another, e.g., using alignment elements (e.g., alignment elements 364, 374), at 406. Optionally, the alignment of the first and second catheters can be confirmed using visualization, markers, or other suitable methods. In some instances, first and second catheters may not be advanced through or positioned within first and second veins and rather a fistula forming device (e.g., first catheter 370) can be used to percutaneously access a cross-over point between the two veins.

At 408, the first and second veins can be pierced (e.g., using piercing element 368 on a first catheter, or using a piercing element advanced through a channel 362 of the first catheter). After forming the opening through the first and second veins, a fistula forming device (e.g., energy delivery source 375) can be advance from the first vein into the second vein, at 410. In some embodiments, the advancement of the fistula forming device can be from the first catheter to the second catheter. For example, a guidewire, shaft, or other structure supporting the fistula forming device can be advanced from the first catheter through the cross-over point between the two veins and into the second catheter (e.g., into a receiving chamber 369 of the second catheter). While advancing the fistula forming device and/or after advancing the fistula forming device, the fistula forming device can be activated to form a fistula via mechanical abrasion and/or energy delivery, at 412. For example, the fistula forming device can be rotated or translated to mechanically abrade the surrounding tissue and/or activated to deliver ablative energy to the surrounding tissue.

Optionally, a plug can be deployed to seal one or more openings in the veins separate from the fluid connection, e.g., to prevent a hemorrhage, at 414. Alternatively or additionally, a hemorrhage can be prevented via delivery of an energy source, suturing, hemostatic agents, or any combination thereof. The first and second catheters (and any other devices used during the procedure) can be removed, at 416.

In some embodiments, blood flow from the renal vein can also be diverted toward the target vessel and/or fluid connection, e.g., to increase a pressure difference across the fistula, at 418. Increasing the pressure difference can improve a success rate of the fistula, e.g., by reducing a likelihood of undesirable clotting.

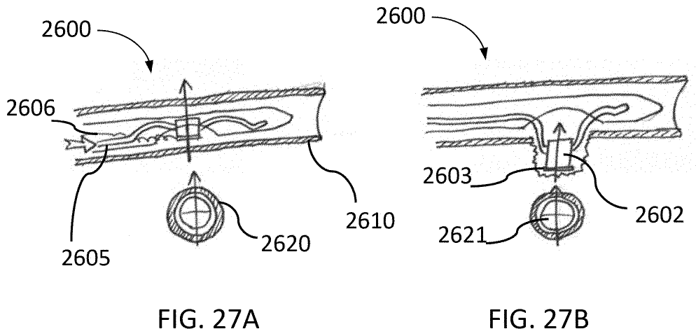

FIG. 26 depicts an example embodiment of a device 2600 for forming a fistula. FIGS. 27A and 27B depict the use of the device 2600 to form a fistula between two blood vessels, 2610 and 2620. As depicted, the source catheter 2601 (e.g., a first catheter) has a self-guided member for fistula creation between two blood vessels at an angle. The self-guided member comprises of an electrode member 2602 (e.g., energy delivery element, such as, for example, an electrode comprised of tungsten, or some other metal) combined to an alignment member 2603 (e.g., alignment element such as, for example, a magnetic material). A layer of insulator can separate the electrode and the alignment members. The combination of the alignment member and electrode is extended radially away from the body of the catheter, using deformable elements 2604. The deformable elements 2604 may be deformed by a mechanical force, as represented by arrow 2605 in FIG. 27A, or other mechanisms (e.g., electrical, magnetic, etc.). The electrodes can be energized by transmission through a conduit 2606 for energy. Activation of the electrode allows for delivery of energy to the surrounding tissue. This energy may be in the form of thermal, electrical, RF, DC, cryogenic, chemical or a combination thereof. The extension of the alignment member enables the tissue to be simultaneously subjected to mechanical energy, required to bridge two blood vessels, while part of the tissue surrounding the electrode is killed.

In one embodiment, the alignment member can be, for example, a magnet and/or an electromagnet to facilitate self-alignment with the target catheter.

The target catheter 2621 may be include a single alignment member or a combination of alignment members 2622. The alignment member 2622 can be, for example, a magnet and/or an electromagnet to facilitate the self-alignment with the source catheter 2601. The alignment member 2622 can guide the electrode member 2602 as the member is extended through the surrounding tissue to create a fistula.

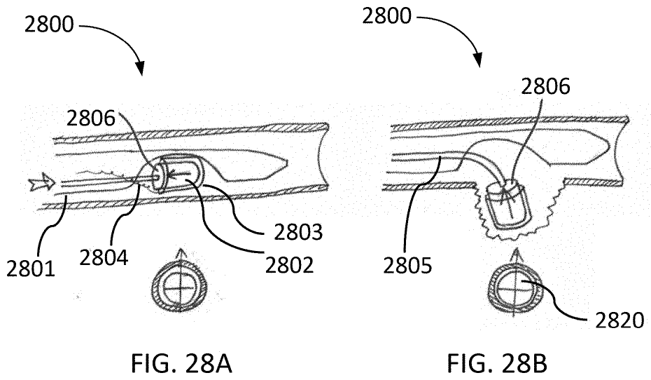

FIGS. 28A-28B depict another example embodiment of a device 2800 for forming a fistula. As depicted, the source catheter 2801 (e.g., first catheter) has the self-guided member 2802 in the form of a cylinder. The alignment member (e.g., alignment element) 2806 comprises of the bulk of the cylinder and is surrounded by a layer of insulation and electrode element 2803 (e.g., energy delivery element). The electrode element 2803 is separately connected to source of energy and has a dedicated channel 2804 (e.g., a conductive wire, flexible circuit, etc.) for transfer of energy from the source (external to the patient), to the electrode element 2803. The alignment member 2806 can be comprised of a magnet or electromagnet or ferroelectric material, actuated by external field. The dielectric layer can be comprised of any insulating material that provides thermal, cryogenic, electric and/or magnetic insulation.

The self-guided member 2802 can be housed inside a recessed cavity in the source catheter 2801. Due to the orientation of the self-guided member 2802, the force of attraction towards the target catheter 2820 may be highest as the self-guided member moves closer to the target catheter 2820. The orientation of the self-guided member 2802 can also change as the member 2802 moves through the interstitial tissue towards the target catheter 2820. Alternatively, the orientation and position of the self-guided member 2802 can be changed by connecting element 2805, which can be manipulated proximally (e.g., using pull wires).

The target catheter 2820 can be structurally and/or functionally similar to the second catheter described with reference to FIG. 24. In some embodiments, the target catheter 2820 configured for use with the source catheter depicted in FIGS. 28A and 28B can be the target catheter 2620 depicted in FIG. 26.

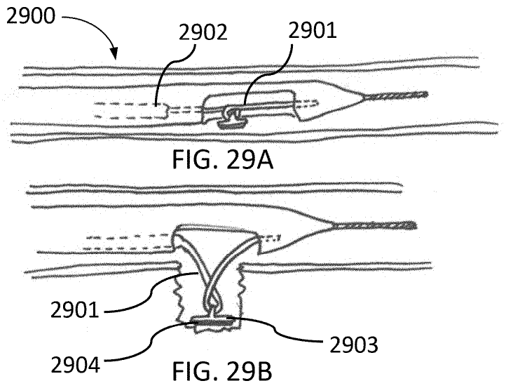

FIGS. 29A-29B depict another example embodiment of a device 2900 for forming a fistula. As depicted, the mechanism to create fistula on the source catheter (e.g., first catheter) can comprise of a deformable member 2901 (e.g., shaft) that can be extended outwards from the catheter body by use of mechanical translation of a connected member 2902 and the deformable member comprises of an electrode member 2904 (e.g., energy delivery element) pressing against the blood vessel walls. As shown in FIGS. 29A-29B, the deformable member is in the form of a torsional spring 2901 that can be extended by translating the proximal extension arm 2902. The distal end of the deformable member is fixed in the distal end of the catheter. The deformable member can be comprised of materials including, but not limited to, metal, alloy, plastic, polymer, or a combination thereof. The deformable member can act as the means of transfer of energy to the electrode member. The electrode is extended outward as the deformable member deforms. The electrode can have a pivot point to enable self-alignment 2903 with the blood vessel walls as it creates fistula. The electrode member can have radiopaque properties or an additional radiopaque marker for visualization under fluoroscopy or x-ray field. The electrode member can also have other self-alignment members 2903 including, but not limited to, magnets, electromagnets, ferroelectrics, to self-orient and align the electrode member and minimize risk. Additionally, there can be another alignment member to indicate the final position of the fully extended member. This can be useful in positioning the source catheter with respect to the target catheter to increase the chances of technical success of the procedure.

The target catheter (not depicted) can be structurally and/or functionally similar to the second catheter described with reference to FIG. 24. In some embodiments, the target catheter configured for use with the source catheter depicted in FIGS. 29A and 29B can be the target catheter depicted in FIG. 26.

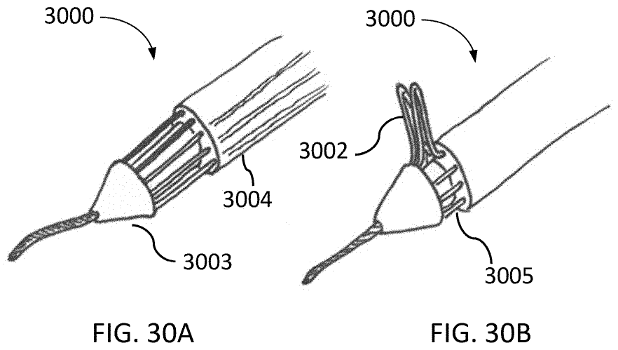

FIGS. 30A-30B depict another example embodiment of a device 3000 for forming a fistula. As depicted, the source catheter 3001 (e.g., first catheter) can comprise of multiple deformable members 3002. FIGS. 30A-30B show multiple deformable members 3002 that are deformed by mechanical translation of the distal end 3003 of the source catheter with respect to the proximal end 3004 of the catheter 3001. There can be alignment members 3005 present to guide the mechanical compression of the deformable members 3002. These alignment members 3005 can ensure that the deformable members 3002 are deforming in the desired direction and prevents any undesired rotation, torque or translation. The deformable members 3002 can comprise of electrode members (e.g., energy delivery elements) built into the deformable members 3002 or can have electrode elements attached to the deformable members 3002.

The source catheter depicted in FIGS. 30A-30B can be used with a target catheter (not depicted) that is structurally and/or functionally similar to the second catheter described with reference to FIG. 24. In some embodiments, the source catheter can be used with the target catheter depicted in FIG. 26

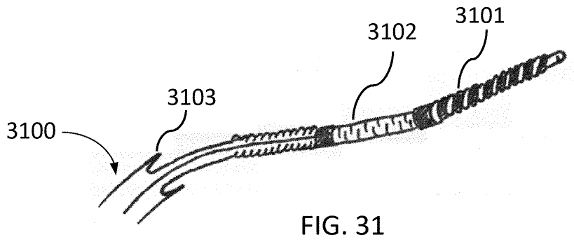

FIG. 31 depicts another example embodiment of a device 3100 for forming a fistula. In some embodiments, the source and target catheters (e.g., first and second catheters) act as hollow channels to translate a device 3100 implemented as a custom shaped guidewire. As shown in FIG. 31, the custom shaped guidewire comprises of a distal tapered conductive tip 3101, a medial segment with embedded electrodes 3102 (e.g., energy delivery element), and a proximal end with sharp edges 3103. The diameter of the proximal end of the guidewire can be about 0.5-5 mm and the distal tip of the guidewire can be in the range of about 0.1-2 mm. The embedded electrode 3102 may comprise of materials capable of delivering thermal, electrical, chemical energy or a combination thereof. Energy maybe delivered to the electrode 3102 either from the proximal end of the guidewire connected to an external source of energy, or from the distal end of the guidewire after it makes connection with a mating element, e.g., snare element 366 as depicted in FIG. 24, in the target catheter, or a combination of the two.

The distal tip of the guidewire can comprise of a recessed notch to align and mate with a snare or ratcheting mechanism (e.g., snare element 366 as depicted in FIG. 24), in the target catheter. This mechanism can provide stability to the system and can act as a mechanism of transferring energy or completing a circuit, where the snare is electrically conductive and connected to an external energy source. This mechanism can allow pulling of the guidewire from the source catheter into the target catheter, e.g., by connecting the snare or ratcheting mechanism to an external pull mechanism on the proximal end of the target catheter. When the guidewire is pulled into the target catheter, the sharp edges can abrade tissue and create a fistula equivalent or substantially equivalent to the proximal diameter of the guidewire.

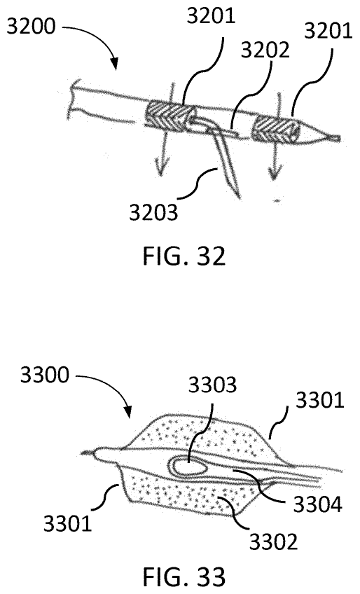

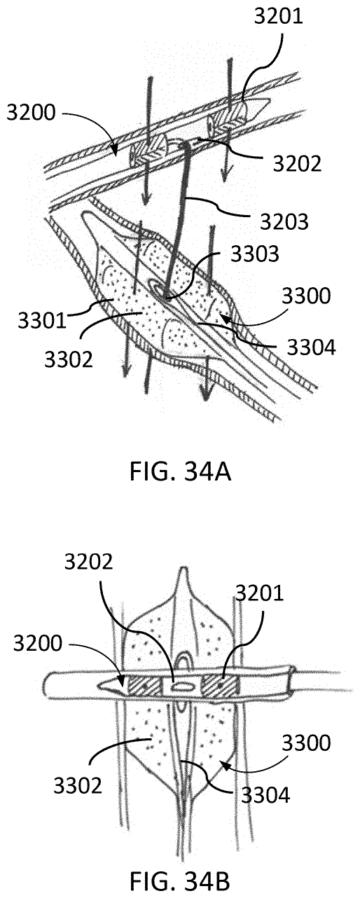

FIGS. 32 and 33 depict another example of a source catheter 3200 (e.g., a first catheter) and a target catheter 3300 (e.g., a second catheter), respectively, according to embodiments described herein. The target catheter 3300 comprises of an expandable element (e.g., a balloon and/or cage) capable of containing magnetic particles. These magnetic particles mate with magnetic elements on the source catheter to help align the two catheters and facilitate cross over of the piercing element, as depicted in FIGS. 34A-34B. The target catheter expandable element can be relatively flat in shape to allow for stretching of the blood vessel and facilitate alignment with the source catheter 3200.

The source catheter 3200 comprises one or more magnetic elements 3201 (e.g., permanent magnets) that are used as alignment members with the target catheter 3300. The source catheter 3200 can also contain a dedicated lumen with a side opening 3202 used to pass a mating element 3203 (e.g., guidewire, needle). The target catheter 3300 comprises of an expandable member 3301 (e.g., a balloon and/or cage) capable of containing magnetic particles 3302. The target catheter 3300 also has a dedicated opening 3303 for capturing the mating element 3203, and a dedicated retrieval mechanism 3304 (e.g., snare element) embedded in one of its lumens.

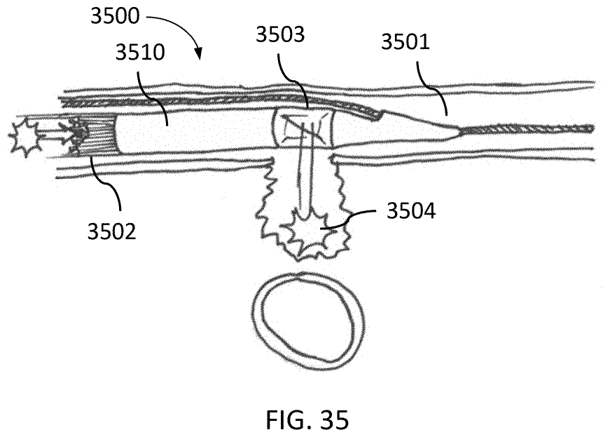

FIG. 35 depicts another example embodiment of a device 3500 for forming a fistula. As depicted, the source catheter 3510 (e.g., first catheter) comprises of an optic channel 3502 to transfer electromagnetic radiation from an external source to the tip of the catheter 3510. Such electromagnetic energy can be in the form of laser, optical light, infra-red radiation, etc., and can be delivered as depicted by 3504. At the tip of the source catheter, there can be a lens, or a system of lenses or prisms 3503 (e.g., energy delivery element) to reflect the energy perpendicular to the axis of the source catheter 3510. There can be an alignment member (not depicted) present on the source catheter to facilitate orientation and/or fixation of the source catheter with respect to the target catheter (e.g., second catheter).

In an embodiment, the device 3500 can include a fiber optic bundle 3502 (e.g., including one or more light guides); a component used to deflect the light source, such as, for example, lens assembly 3503; and the light source 3504 configured to generate the energy used to ablate target tissue (e.g., a laser). In some embodiments, the example embodied in the device 3500 can include a rapid exchange tip 3501, e.g., for trackability. While not specifically depicted with respect to other devices described herein, any of the other devices for forming fluid connections and/or fistulas can include a rapid exchange tip, such as, for example, rapid exchange tip 3501, e.g., to save lumen space. Alternatively, the device 3500 can comprise of a regular guidewire lumen.

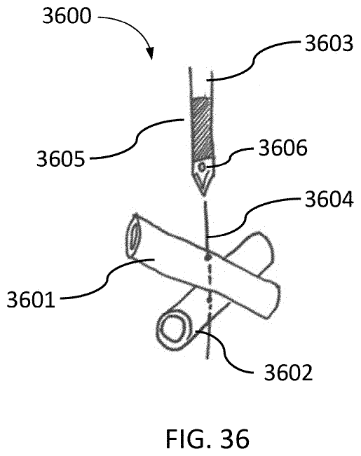

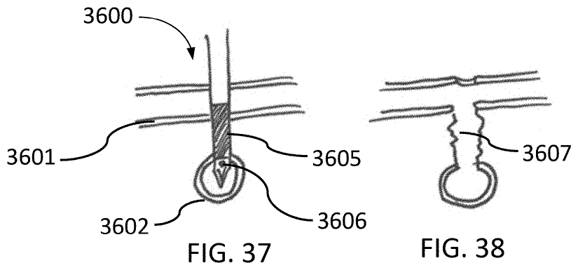

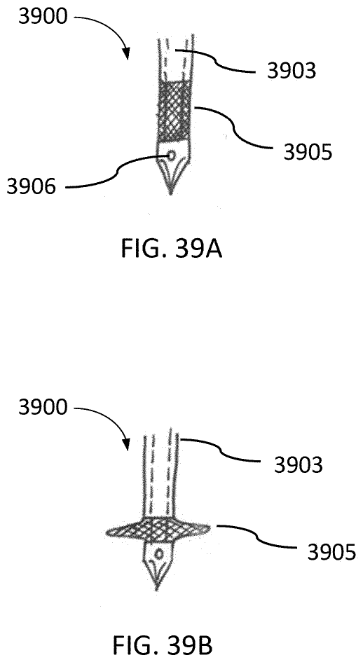

FIGS. 36 and 39A-39B depict other example embodiments of devices 3600, 3900 for forming a fistula. As depicted, the formation of a fistula can be achieved via a percutaneous approach using imaging guidance. As an illustrative example, the percutaneous deployment of the devices 3600, 3900 is summarized by the graphical sequence summarized in FIGS. 37-38 and 40A-40C, respectively.

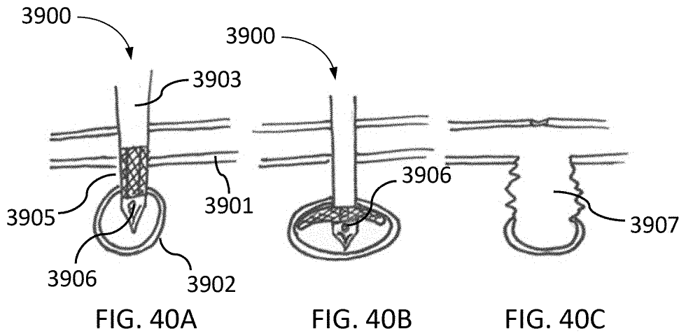

An example percutaneous approach for the creation of a fluid connection between two blood vessels, e.g., vessels 3601 and 3602 as depicted in FIGS. 37-38, or vessels 3901 and 3902 as depicted in FIGS. 40A-40C, with a fistula through a percutaneous approach may comprise the following steps.

First, based on patient-specific anatomical data, a target pathway, 3604, through the body is determined. The anatomical data for the patient may be obtained using, but not limited to, ultrasound, magnetic resonance imaging, computed tomography, doppler, optical imaging, fluoroscopy, radiography, or any combination thereof. The determined pathway insects with, at least in part, a first blood vessel, 3601 or 3901, and a second blood vessel, 3602 or 3902, of which at least one blood vessel comprises the spermatic vein. There are multiple possible, adequate, and/or clinically relevant pathways that the determined pathway may comprise. An exemplary ideal trajectory may intersect with the two target blood vessels while avoiding any critical anatomical structures.

A bridging member, 3603 or 3903 (e.g., first catheter 370), which may comprise any suitable structure including, but not limited to, a needle, a probe, a catheter, or a minimally invasive delivery tool, is advanced along the determined pathway. The bridging member may comprise position sensors that determine the position of the bridging member in three-dimensional space in real-time. The bridging member may also comprise of a mechanism to confirm properties of surrounding tissue or structures. There can be multiple such members distributed throughout the body of the device to identify the various structures, for example simultaneous determination of distal blood vessel and proximal blood vessel. This can be helpful in determining whether the bridging member is located within blood vessel space or interstitial tissue space. In one example, this can be accomplished by delivering a bolus of fluid through an orifice, 3606 or 3906, disposed at a distal end of the bridging member. The advancement of the bridging member may be guided in real-time based on the patient's anatomical data and the position of the bridging member in three-dimensional space with respect to reference markers. The reference markers may comprise anatomical structures, fiducial markers, bony structures, or any other suitable marker. The advancement of the bridging member can be performed by a robotically-assisted device or system. In some embodiments, there can be a member located inside the proximal blood vessel that acts as a hard stop (not shown) for the distal end (or the electrode carrying member) as it approaches close to the proximal blood vessel.

Once the bridging member, 3603 or 3903, is in the desired location, an electrode or electrode-carrying members, 3605 or 3905 (e.g., components of a first catheter 370 supporting energy delivery elements 375), can be deployed or activated. As shown in FIGS. 36-40B, the electrode carrying members can be attached to the main body of the bridging member, 3600 or 3900, at multiple points. As shown in FIGS. 36-38, when the electrode carrying member is connected at more than two locations, the electrode carrying member remains flush with the body of the bridging member, 3600. In some embodiments, the electrode carrying members can have rough surface to facilitate tissue debridement post tissue treatment. The electrode and electrode-carrying members, 3605 or 3905, may be comprised of, for example, metal, alloys, plastic, composites, polymer, gel, conductive polymers, conductive gel, or a combination thereof. The electrode-carrying members, 3605 or 3905, provide mechanical support to the electrode members (e.g., energy delivery elements 375) and a mechanism to transfer energy from an external source (e.g., one or more conductive wires). The electrodes deliver energy to the surrounding tissue for formation of a fistula, 3607 or 3907.

In some embodiments, from FIGS. 39A and 39B, when the electrode-carrying member, 3905, is in the desired location, the electrodes can be deployed to extend the electrodes and electrode-carrying members, 3905. This can be accomplished by mechanical translation of the distal tip towards the proximal body of the bridging member. Since the electrode-carrying member, 3905, is connected to the proximal end of the distal tip and to the distal end of the proximal body, the electrode-carrying member, 3905, expands outwards as the two attached ends are pushed closer to each other.

While the electrodes deliver energy to the surrounding tissue, electrode-carrying members or electrodes, 3605 or 3905, can be shaped to facilitate debridement of treated tissue. One example of accomplishing this is simultaneous movement of the device along with delivery of energy through the electrodes.

The bridging member, 3603 or 3903, may optionally comprise a mechanism for preventing hemorrhage of at least one target blood vessel prior to complete removal of the bridging member, 3603 or 3903. The mechanism for preventing hemorrhage may comprise deploying a blood vessel plug (e.g., as depicted in FIG. 15A), delivery of an energy source, suturing, hemostatic agents, or any combination thereof. Alternatively, at least one of the target blood vessels may be closed using external pressure or a pre-shaped shunt that extends through the lumen of the first blood vessel, as depicted in FIG. 15B.

### 3. Renal Vein Flow Rate Adjustment

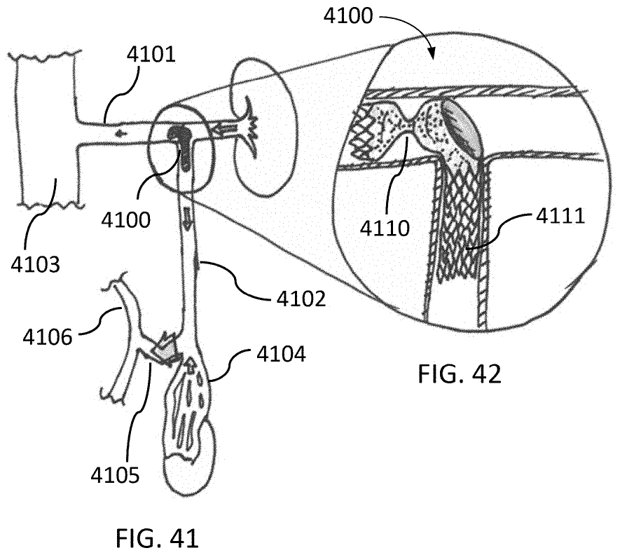

Due to low flow rates in a venous system, foreign body implants, shunts, or fistulas are prone to patency issues from undesirable clotting. In order to increase success rates, flow rate can be adjusting by increasing the pressure difference across the implant, shunt or the fistula. The pressure difference can be changed by varying the pressure and/or flow rates at the inlet of the implant or fistula, outlet of the implant or fistula, or both. From FIG. 41, the flow rate through a fistula or segment 4105 can be increased by increasing the flow rate from the gonadal vein 4102 and 4104, through the use of an implantable device 4100 that diverts additional blood into the gonadal vein 4102 from the left renal vein 4101.

FIGS. 41 and 42 depict an example embodiment of using an endovascular stent 4100 to divert blood away from a renal vein 4101 toward the gonadal vein 4102 (and therefore the spermatic vein). As depicted, an endovascular stent can be placed in the left renal vein and effectively reduce the cross-sectional diameter of the left renal vein 4101 between the gonadal vein 4102 and the inferior vena cava 4103. This can be accomplished using a tapered covered stent 4100, as shown in FIGS. 41 and 42. As a result of the reduced cross-sectional area, higher volume of venous blood from left kidney can be directed towards the gonadal vein 4102, as indicated by the arrows. The size of the arrows qualitatively represent flow rates in FIG. 41.

In some embodiments, the endovascular device can have a curved or extended portion 4111 that attaches to the gonadal vein. This can prevent accidental migration of the device 4100 away from the left renal vein. The device 4100 can be a stent with body comprising of, for example, metal, plastic, polymer, alloy, or a combination thereof. The device 4100 can be covered completely or partly, with the covering comprising of, for example, plastic, polymer, stretched polymer, nanofiber matrix, gel, tissue, 3D printed material or a combination thereof.

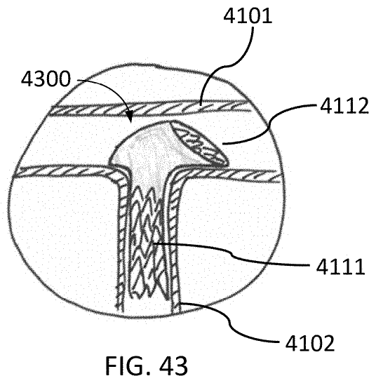

FIG. 43 depicts an example of another endovascular device including a flow diverter 4300 that diverts a portion of the venous blood flow in the left renal vein 4101 to the gonadal vein 4102. The increased cross section of the flow diverter 4112, as compared to the diameter of the gonadal vein 4102, redirects a portion of the renal venous output towards the gonadal vein 4102. A portion or all of the endovascular device 4300 can be covered to facilitate flow redirection.

In some embodiments, the endovascular devices 4100, 4300 can have radiopaque markers on the body for ease of orientation and deployment. In some embodiments, the endovascular devices 4100, 4300 can have flared ends to sit flush with a blood vessel opening. In some embodiments, the endovascular devices 4100, 4300 can have hooks or piercing elements, e.g., in the 4111 portion that engages with gonadal vein 4102, to prevent migration post-deployment.

The devices 4100, 4300 can be delivered and deployed using a percutaneous or laparoscopic delivery device.

### Systems, Devices, and Methods for Occlusion or Ligation of a Target Blood Vessel

Set forth below is a detailed description of various embodiments of a medical device and method for occlusion or ligation of a target blood vessel to aid in the treatment of varicocele, erectile dysfunction, infertility, nutcracker syndrome, benign prostate hyperplasia (BPH), bladder cancer, prostate cancer, pelvic congestion, ovarian cancer, polycystic ovarian syndrome, uterine fibroids, endometriosis and/or hormonal disorders. Such devices and methods can provide more effective treatments to reduce, eliminate, and/or prevent pathophysiological hydrostatic pressure in the testicular or ovarian venous drainage system.

In particular, systems, devices, and methods disclosed herein can reduce or eliminate the pressure gradient between the testicular drainage systems, preventing back-flow of blood from the testes to the prostate. The normal physiological functioning of the prostate is restored, with normal venous pressures, a normal arterial blood flow through the prostate, and exposure of the prostate to normal levels of free testosterone.

Systems, devices, and methods described herein also provide approaches and improvements to ligation and/or occlusion of the spermatic or ovarian vein(s) (or other blood vessels) to eliminate the pathophysiological hydrostatic pressure from faulty, malfunctioning one-way valves in the ISV and/or the accompanying network of retroperitoneal venous bypasses. These methods enable ligation and/or occlusion of the network of venous bypasses regardless of vessel diameter. Elimination or reduction of the pathological hydrostatic pressure by these treatments restores normal arterial oxygenated blood flow and normal supply of nutrient materials to the seminiferous tubules, i.e., the sperms' production site.

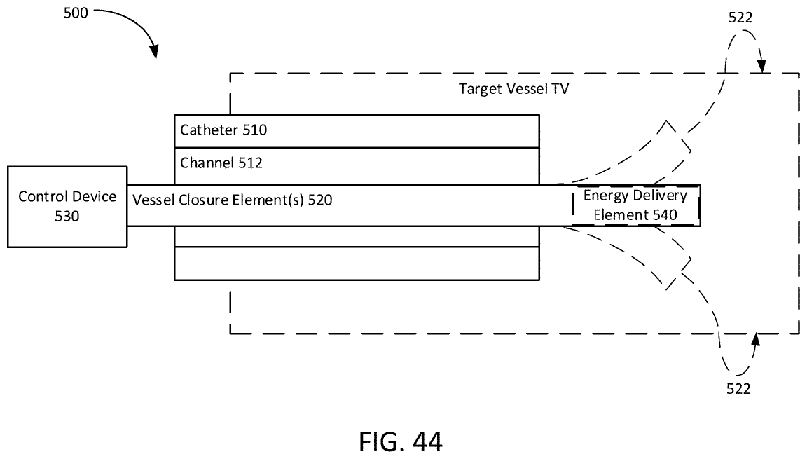

FIG. 44 schematically depicts an example device 500 for ligating and/or occluding a target vessel TV. The device 500 can include a catheter 510 that defines a channel 512. The channel 512 can be a working channel that receives and/or houses one or more vessel closure element(s) 520. In some embodiments, the channel 512 can be configured to receive other medical components, e.g., a visualization mechanism (e.g., lens, light source, etc.), sensors, etc. In some embodiments, the channel 512 can be configured to deliver a fluid such as an agent (e.g., a contrast agent), chemical, therapeutic substance, anesthetic, etc. into the target vessel.

The vessel closure element(s) 520 can be configured to cause occlusion and/or ligation of the target vessel TV. In some embodiments, the vessel closure element(s) 520 can optionally include energy delivery element(s) 540, such as, for example, electrode(s) and/or other energy conducting element(s) that can deliver energy to tissue. The energy can include, for example, thermal, electrical, RF, DC, cryogenic, chemical, or a combination thereof. In some embodiments, the delivery of energy to tissue can cause ablation and/or embolization of tissue, which can lead to vessel closure. In some embodiments, the vessel closure element(s) 520 can optionally include one or more mechanical components, e.g., a spring, an arm, a clamp, a shaft, etc. Such mechanical component(s) can be configured to move and/or deform to grasp and/or close around the target vessel, as illustrated by 522. In some embodiments, a vessel closure element 520 can be formed of shape memory material that can return to a predefined shape in response to certain conditions. For example, a shape memory vessel closure element 520 can be configured to return to a compressed state to close the target vessel TV in response to being exposed to body heat.

In some embodiments, the device 500 can include a control device 530 that can control delivery of energy to components of the device 500 (e.g., energy delivery element 540) and/or control deployment and/or movement of the vessel closure element(s) 520. In some embodiments, the control device 530 can be a knob or handle that allows a user to mechanically manipulate one or more pull wires to movement a vessel closure element 520. In some embodiments, the control device 530 can be an electronic controller including a processor that is configured to automatically move a vessel closure element 520 and/or activate delivery of energy to an energy delivery element 540.

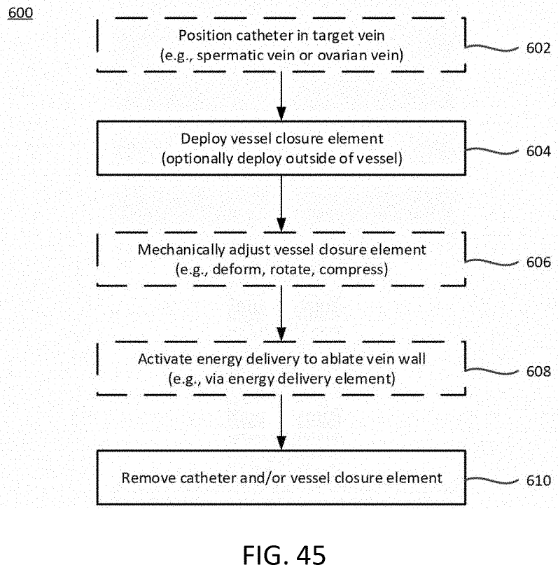

FIG. 45 depicts an example method 600 of occluding and/or ligating a target vessel. A catheter (e.g., catheter 510) an optionally be positioned within patient anatomy, at 602. In instances involving an endovascular approach, the catheter can be positioned within a target vein (e.g., a spermatic or ovarian vein). In instances involving a percutaneous approach, the catheter can be inserted into tissue into a target vein. At 604, one or more vessel closure element(s) (e.g., vessel closure element(s) 520) can be deployed. Optionally, in some instances, the vessel closure element(s) can be deployed such that at least a portion of the vessel closure element(s) are positioned external to the target vein. In some embodiments, the vessel closure element(s) can include piercing and/or puncturing elements for puncturing through a wall of the target vein. Alternatively, the catheter can include a piercing and/or puncturing element for puncturing through a wall of the target vein such that a vessel closure element can be extended through the opening and around the target vessel.

Optionally, at 606, the vessel closure element(s) can be mechanically adjusted or manipulated to close around a vessel. In some embodiments, a vessel closure element can be formed of shape memory material that can revert back into a predetermined shape in response to certain conditions (e.g., body heat). As such, once such vessel closure element is delivered into the patient anatomy, it can automatically change shape to close around and/or occlude the target vein. Optionally, at 608, the vessel closure element(s) can include one or more energy delivery element(s) (e.g., energy delivery element 540), which can be activated to deliver energy to surrounding tissue, e.g., to ablate and/or cause embolization of such tissue.

The catheter, vessel closure element, and other components can be removed after the vessel is occluded and/or ligated, at 610.

### 1. Internal Ablation of Blood Vessels to Induce Embolization

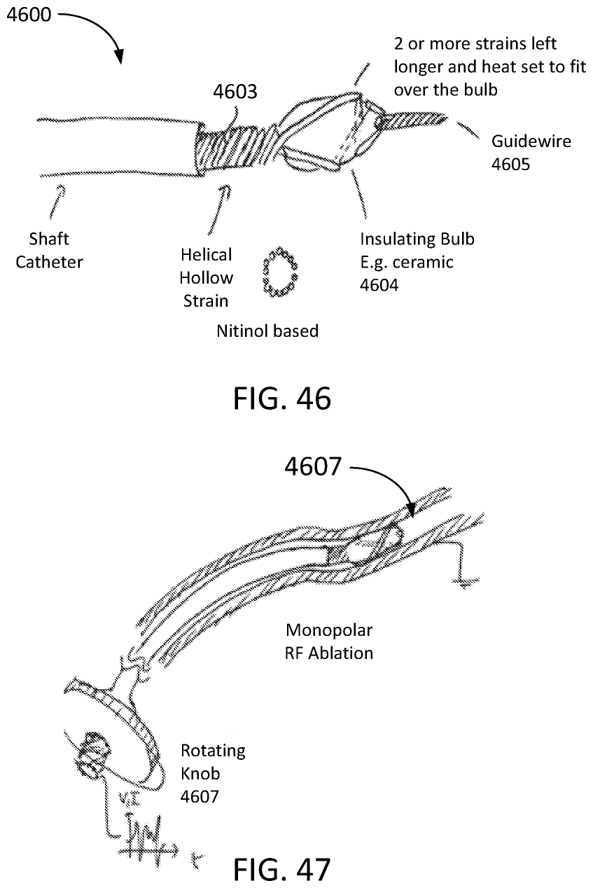

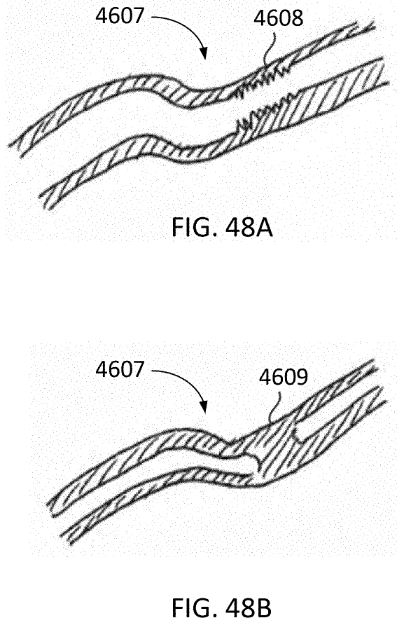

FIGS. 46 and 47 depict an example catheter 4600 comprising a helical hollow strain (HHS) shaft 4603, which may additionally comprise a shape memory alloy. The ablation catheter shaft 4603 may comprise a central lumen configured for advancement over a guidewire 4605, and a flexible and highly torquable shaft, with two or more distal, longer, heat-set, strains around a bulb shaped mandrel 4604. The distal strains may be electrically connected (e.g. via motor brushes) to an energy source such as a radio frequency generator. The distal tip of the ablation catheter is coupled with the insulating bulb 4604 and comprises a material with high dielectric constant (e.g. ceramic, PET, PEEK).

The ablation catheter 4600 can be configured to ablate the inner layers of a target vessel 4607 to induce embolization and closure of the target vessel by activating the delivery of energy to the ablation bulb while rotating the bulb. The ablation catheter 4600 can include a control device implemented as a rotating knob 4606 that can be used to move (e.g., translate and/or rotate) the strains and/or insulating bulb, e.g., to facilitate inducing embolization and closure of the target vessel.

Once the ablation catheter 4600 has damaged the intimal layer of the target vessel 4608, the inflammatory response can ensure closure 4609 of the controllably damaged vessel.

### 2. Heat-Activated Spring for Vessel Closure

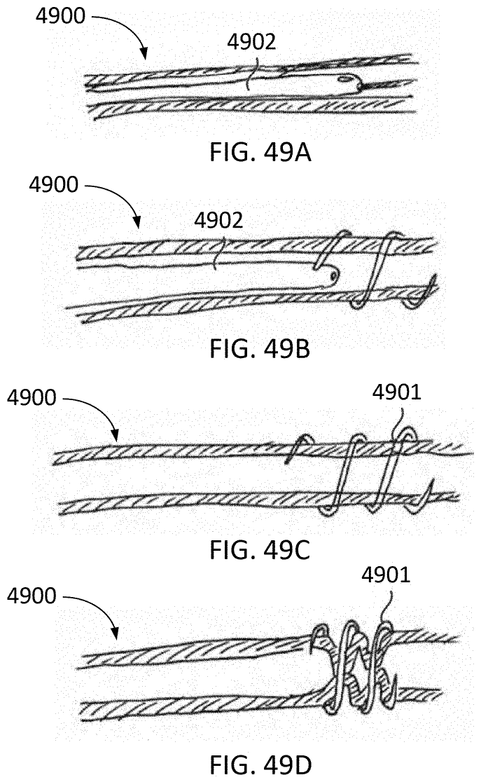

FIGS. 49A-49D depict an approach for closing a target vessel using a different example device 4900 (e.g., a catheter). The device 4900 can be implemented as a spring catheter 4900. The spring catheter 4900 comprises a heat-set spring 4901 that further comprises a shape memory alloy configured to generate axial compression forces on a target vessel. Precise heat-setting property of the spring is performed so that the shape of the heat-set spring is automatically reshaped when the material reaches a predefined temperature, e.g., about 37° C. (body temperature). In some embodiments, the heat-set spring 4901 may comprise a shape memory alloy with an austenitic phase transition temperature between about 34 and about 37° C.

By nesting the spring in a cooled delivery system 4902 in a straight configuration, as depicted in FIG. 49A, the heat-set spring 4901 can be deployed from the inside of a target vessel, to spiral outside of the vessel with a pitch, e.g., determined by the deployment channel included in the delivery system tip. The “stretched” spring 4901 surrounding the vessel can return to its heat-set, compressed shape when exposed to body temperature. In turn, the deployed spring 4901 exerts axial compression forces on the vessel, as depicted in FIG. 49D, causing the target vessel to collapse and close on itself

### 3. Intraluminal Vessel Ligation Catheter

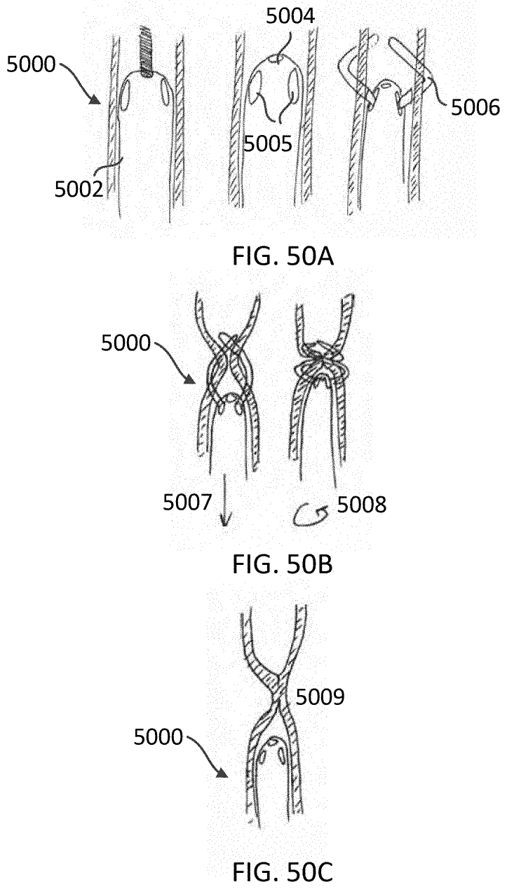

FIGS. 50A-50C depict an approach for sealing and closing a target vessel using another example device 5000 (e.g., a catheter). A vessel ligation catheter 5000 comprises a multi-lumen catheter shaft 5002, a distal tip 5004 comprising a material with high dielectric constant (e.g., ceramic, PEEK), a central lumen configured for advancement of the vessel ligation catheter over a guidewire, and at least two side apertures 5005 configured for the advancement of two or more ligation members 5006 (e.g., vessel closure elements).

The ligation members 5006 reside within the shaft 5002 of the ligation catheter 5000 and comprise a shape memory alloy (for example, Nitinol). The ligation members 5006 are configured to puncture through the wall of a target blood vessel by advancing the wires out of the side apertures 5005 on the ligation catheter 5000. In one example embodiment, the ligation members are pre-shaped in a coiled configuration, and therefore spiral around the blood vessel after puncture. The ligation members 5006 may comprise a connection to an energy source, such as, for example, radiofrequency energy, to assist in puncture of the blood vessel wall.

Retracting 5007 the ligation members and/or applying torque 5008 to the ligation catheter 5000 can result in mechanically twisting, or collapsing, of the vessel, thereby reducing or completely eliminating blood flow therethrough. The ligation members 5006 may comprise a connection to a radiofrequency generator or other energy source. Through the activation and delivery of radiofrequency energy through the ligation members 5006 to the blood vessel, the twisted, or collapsed, blood vessel is ablated and closed. In some embodiments, the radiofrequency energy source may comprise at least one monopolar electrode and a grounding pad placed onto the patient's body. In some embodiments, the radiofrequency energy source may comprise at least two ligation members 5006 configured for bipolar ablation of the blood vessel (and thus would not require a grounding pad placed onto the patient's body).

### 4. Percutaneous Vessel Sealing Device

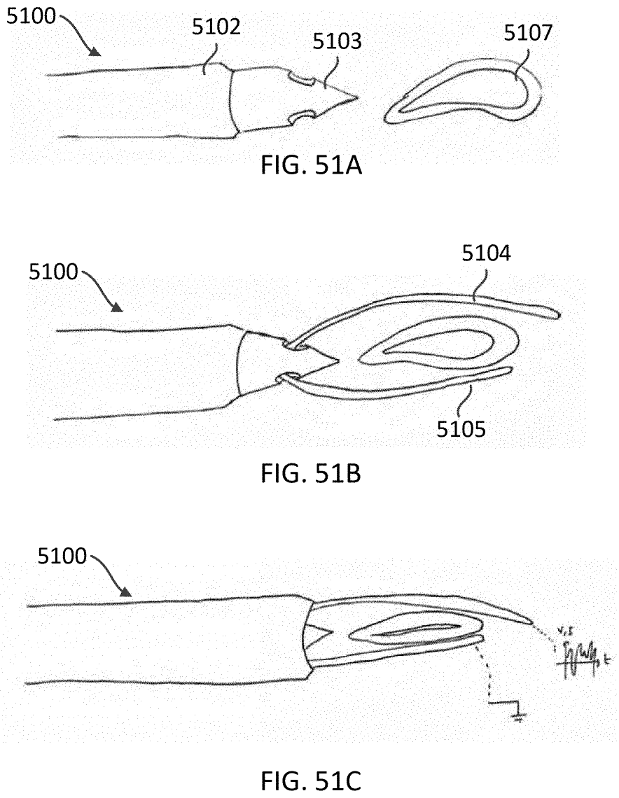

FIGS. 51A-51C depict a process of using another example device 5100 (e.g., catheter) to seal and close a target vessel to prevent blood flow. The vessel sealing device 5100 is introduced into the body via a percutaneous, or minimally invasive, approach. The vessel sealing device 5100 can include a distal end 5103 that includes openings for extending one or more sealing members. The vessel sealing device 5100 comprises at least two sealing members 5104 and 5105 (e.g., vessel closure elements) that are configured to encompass a target blood vessel 5107, in this particular example the spermatic vein(s), and deliver an energy to seal the vessel to prevent the flow of blood therethrough.

The sealing members 5104 and 5105 may comprise a straight or curved shape and can be reconfigured for deployment from the distal tip of the vessel sealing device 1000. The sealing members 5104, 5105 may comprise a shape memory alloy such as nitinol.

An example method for the deployment and use of the vessel sealing device 5100 to reduce or eliminate blood flow through a blood vessel (such as, for example, the spermatic (or testicular) vein, spermatic (or testicular) artery, ovarian vein, ovarian artery, deferential vein, deferential artery, cremasteric artery, and/or the cremasteric vein) may comprise the following steps.

First, using any suitable imaging modality including, but not limited to, ultrasound, fluoroscopy, electromagnetic imaging, or any combination thereof, the vessel sealing device 5100 (e.g., catheter) is percutaneously introduced in close proximity to the target vessel. Once the distal tip of the vessel sealing device is properly placed, the sealing members 5104, 5105 are advanced through the shaft of the vessel sealing device to encompass the target blood vessel 5107.

The sealing members are then closely approximated to apply a compressive force to the vessel and, in one particular embodiment, the mechanism for approximating the sealing members 5104 and 5105 may comprise the advancement of a camming member 5102 to push the sealing members together. The target blood vessel 5107 is occluded at least in-part through the approximation of the sealing members 5104 and 5105, and the blood vessel is sealed through the activation and delivery of an energy source. The energy source may comprise, for example, radiofrequency energy.

After sealing the target blood vessel, the vessel sealing device 5100 can be removed.

### 5. Method for the Embolization of a Target Blood Vessel for the Treatment of Varicocele

In another exemplary method for the treatment of, at least partially, varicocele, erectile dysfunction, infertility, nutcracker syndrome, BPH, bladder cancer, prostate cancer, pelvic congestion, ovarian cancer, polycystic ovarian syndrome, and/or hormonal disorders, certain blood vessels can be ligated or occluded. These blood vessels may include the following blood vessels and any branching blood vessels that terminate in the following blood vessels: spermatic (or testicular) vein, spermatic (or testicular) artery, ovarian vein, ovarian artery, deferential vein, deferential artery, cremasteric artery, cremasteric vein, etc.

The ligation and/or occlusion of at least one these blood vessels, or any combination thereof, can be achieved using a device that delivers an occluding agent. The device for the delivery of an occluding agent may comprise a catheter (e.g., catheter 510), microcatheter, laparoscopic tool, needle, or any other suitable configuration thereof. The delivery device may comprise an inflatable balloon to anchor the device in place and/or aid in the delivery of the occluding agent. In one particular embodiment, the delivery device comprises a catheter (e.g., catheter 510) with a lumen therethrough configured for navigation, advancement, and/or placement over a guidewire. The catheter may further comprise a lumen therethrough configured for the delivery of an occluding agent.

The occluding agent may comprise any suitable agent configured for permanent or transient occlusion of a blood vessel including, but not limited to, one or more embolization coils, a polymer, a hydrogel, an adhesive, a glue, a light- and/or heat-curable material, cyanoacrylates, fibrin based polymers, thrombogenic compounds, devices that release thrombogenic compounds, an expandable member. The occluding agent may comprise metal, an alloy, plastic, a polymer, a hydrogel, a gelatinous material, a microporous material, and/or a material that changes one or more properties via heat, temperature, sound, light, radiation, or any combination thereof.

In an example embodiment, one or more embolization coils are deployed in at least one target blood vessel. The coils induce thrombogenesis and blood clot formation, leading to occlusion, at least in-part, of the target blood vessel. While spermatic vein ligation or occlusion is a common method of treatment, occlusion, at least in-part, of the spermatic artery is another suitable method of treatment.

Occlusion or embolization of the prostate artery is a well-established procedure with high success rate. Occlusion of prostate artery reduces or completely eliminates the supply of blood and nutrients to the prostate, preventing growth and potentially reducing prostate enlargement. Similarly, occlusion of at least one spermatic artery leads to hypotrophy of the testes, which in turn leads to reduction in venous blood outflow and reflux and testosterone production. Reduction in venous blood reflux has been shown to cause a reduction in prostate size. Similarly, exposure of the prostate to testosterone-rich blood has been shown to induce hypertrophy. Some of the advantages of embolizing or occluding at least one spermatic artery (compared to occlusion of the prostate artery or spermatic vein) is easier vessel access via a percutaneous or minimally-invasive approach due to its larger diameter. Furthermore, occlusion of the spermatic artery does not pose the same challenges of access, visualization, occlusion, and/or ligation of collateral blood vessels that are present for closing the spermatic vein(s).

### Replacement Venous Valves

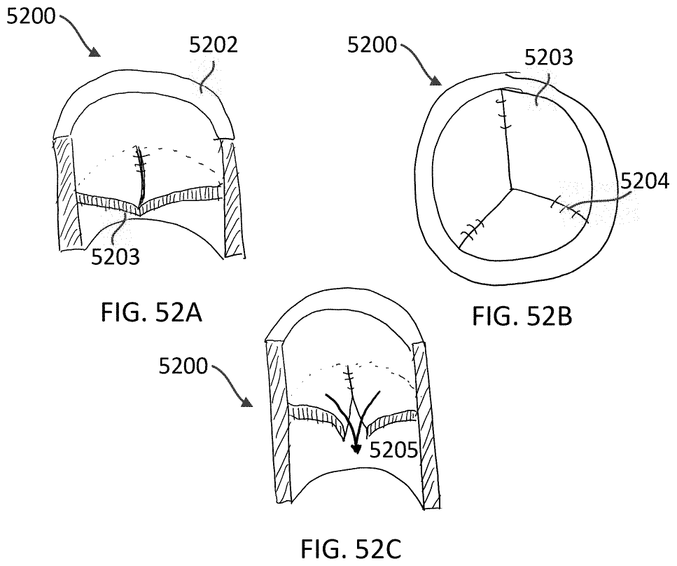

FIGS. 52A-52C depict an example device 5200 for improving blood flow through the testicular venous drainage system. The device comprises an implantable valve configured to replicate the function, at least in-part, of one-way valves in the spermatic vein. The implantable valve 5200 may be introduced into the body via a variety of methods including, but not limited to, a percutaneous or minimally invasive approach.

The implantable valve 5200 is configured to prevent back-flow of venous blood in the testicular venous vasculature after implantation into at least one blood vessel. The implantable valve 5200 comprises an expandable body 5202 configured to securely contact with intima of a blood vessel. The implantable valve 5200 may further comprise one or more foldable flaps 5203 configured to allow for fluid-flow in only one direction. During fluid flow through the valve, at least one foldable flap can be deflected in the direction of the fluid flow 5205. At least one implantable valve 5200 can be placed into a target blood vessel, such as the spermatic vein, and the implantable valve may be placed over a natural one-way valve, proximal to a natural valve, or anywhere along the length of the target blood vessel.

Multiple units of the implantable valve 5200 may be placed along the length of the target blood vessel to reduce hydrostatic blood pressure and/or improve testicular blood flow. The implantable valve 5200 may comprise an outer diameter of about at least one millimeter, and may additionally comprise any suitable biocompatible material including, but not limited to, metal, plastic, polymer, biological tissue, graft, synthetic fibers, gel, or any combination thereof. The foldable flaps 5203 may be attached to the implantable valve body 5202 and/or to each other using any suitable means of attachment 5204 including, but not limited to, sutures, adhesive, curable gel, curable polymer, heat-based welding, or any combination thereof.

While the use of embodiments of the disclosure as set forth herein is described as they pertain to the spermatic vein(s), it can be appreciated that the use of any of the embodiments of the disclosure described herein similarly applies to the ovarian vein(s). Additionally, the use of systems, devices, and methods described herein is bilaterally applicable and is not restricted to a single spermatic or ovarian vein. It can be appreciated that systems, devices, and methods can also be adapted for other patient anatomy, e.g., patient anatomy other than the spermatic and ovarian veins including, for example, vessels in the other vessels in the urinary-tract system, other vessels in the cardiovascular system, other vessels in the brain, other vessels in the legs, arms, or other locations within patient anatomy, etc.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

1. A system for forming a fluid connection between first and second vessels, the system comprising:
a first catheter defining a first channel that terminates in a first aperture, the first catheter including a first alignment element, the first catheter configured to be disposed in the first vessel;
a second catheter defining a second channel that terminates in a second aperture, the second catheter including a second alignment element, the second catheter configured to be disposed in the second vessel, the first and second alignment elements configured to align the first and second apertures of the first and second catheters when the first and second catheters are disposed in first and second vessels;
at least one piercing element configured to pierce through tissue adjacent to the first and second apertures in a space between the first and second catheters; and
a bridging device advanceable through the first channel of the first catheter, through the first and second apertures and the adjacent tissue, and into the second channel of the second catheter such that a portion of the bridging device extends through the space between the first and second catheters, the bridging device configured to allow an implant to be deployed in the space between the first and second catheters to form the fluid connection between the first and second vessel.

2. The system of claim 1, wherein the first vessel is one of: a testicular vein or an ovarian vein.

3. The system of claim 1, further comprising a stopping element configured to prevent advancement of the bridging device beyond a predetermined distance into the second channel.

4. The system of claim 1, wherein the second catheter includes a snare element configured to catch the bridging device and guide the bridging device into the second channel.

5. The system of claim 1, further comprising the implant, the implant including at least one of: a coating for promoting tissue in-growth, or a coating for preventing occlusion in the fluid connection.

6. The system of claim 1, further comprising
an energy delivery element supported by the first catheter, the energy delivery element configured to deliver energy to ablate tissue adjacent to the aperture between the first and second catheters.

7. A method for treating varicocele and associated conditions or any venous insufficiency, the method comprising:
advancing a first catheter into a first vein;
advancing a second catheter into a second vein;
aligning a first aperture of the first catheter with a second aperture of the second catheter;
forming an opening in a wall of the first vein adjacent to the first aperture and an opening in a wall of the second vein adjacent to the second aperture;
advancing a bridging device from the first catheter, through the openings, and into the second catheter such that a portion of the bridging device extends between the first and second veins; and
deploying an implant around the portion of the bridging device extending between the first and second veins.

8. The method of claim 7, wherein the first vein is one of: a spermatic vein, or an ovarian vein.

9. The method of claim 7, wherein the second vein is one of: a deep circumflex vein, a femoral vein, iliac vein, or an epigastric vein.

10. The method of claim 7, further comprising:
activating an energy delivery element to ablate a portion of a wall of the vein to cause closure of the vein.

11.-15. (canceled)

16. A method of treating a clinical urological problem, the method comprising:
dissecting a first vein near the peritoneum; and
anastomosing the first vein to a second vein.

17. The method of claim 16, wherein the first vein includes a spermatic vein or an ovarian vein.

18. The method of claim 17, wherein the second vein includes an inferior epigastric vein, a saphenous vein, a deep circumflex vein, a superior epigastric vein, an iliac vein, inferior vena cava, or a femoral vein.

19. The method of claim 16, wherein the clinical urological problem includes at least one of Benign Prostate hyperplasia (BPH), a lower urinary tract symptom (LUTS), or prostate cancer.

20. The method of claim 19, further comprising:
ligating at least one of the first vein or a third vein.

21. The method of claim 16, wherein the clinical urological problem includes at least one of endometriosis, uterine fibroids, low testosterone, hypogonadism, alopecia, male pattern baldness, Nutcracker syndrome, or infertility.
